(19) United States
(12) Patent Application Publication
(10) Pub. No.: US 2025/0037328 A1
(43) Pub. Date: Jan. 30, 2025

(54) OPTIMIZED VISUALIZATION IN MEDICAL IMAGES BASED ON CONTRAST LEVEL AND SPATIAL LOCATION

(71) Applicant: GE Precision Healthcare LLC, Waukesha, WI (US)

(72) Inventors: Elizabeth Janus Nett, Waukesha, WI (US); Prakhar Prakash, Waukesha, WI (US); Sandeep Dutta, Celebration, FL (US); Michail Fanariotis, Skien (NO); Chuck Bisordi, Oconomowoc, WI (US)

(21) Appl. No.: 18/359,779

(22) Filed: Jul. 26, 2023

(51) Int. Cl. G06T 11/00; A61B 6/00; G06T 5/00; G06T 7/00; G06T 7/11; G16H 15/00; G16H 30/40; G16H 40/63

(52) U.S. Cl. CPC G06T 11/008; A61B 6/463; A61B 6/481; A61B 6/482; G06T 5/90; G06T 7/0016; G06T 7/11; G06T 11/001; G16H 15/00; G16H 30/40; G16H 40/63; G06T 2207/10081; G06T 2210/41

(57) ABSTRACT

Systems and methods are provided for increasing a quality of images generated by a computed tomography (CT) system. In one example, an initial assessment of contrast timing and flow through different anatomical regions of a patient is performed, and based on the initial assessment, different visualization schemes are applied to the different anatomical regions of a reconstructed image, where each visualization is optimized for assessing a different anatomical region. The visualizations may increase a contrast between diseased tissues and non-diseased tissues of the different anatomical regions, and may include color maps (e.g., heat maps and/or probability maps) and/or color overlays based on spectral decomposition information. The different visualizations may be combined into a single 2D image, where each anatomical region (e.g., organ, bone, etc.) is displayed in high contrast, and where aspects of various anatomical regions may be highlighted or colorized to visualize specific information.

### TECHNICAL FIELD

Embodiments of the subject matter disclosed herein relate to non-invasive diagnostic imaging, and more particularly, to real-time adaptive contrast imaging.

### BACKGROUND

Non-invasive imaging technologies allow images of the internal structures of a patient or object to be obtained without performing an invasive procedure on the patient or object. In particular, technologies such as computed tomography (CT) use various physical principles, such as the differential transmission of x-rays through the target volume, to acquire image data and to construct tomographic images (e.g., three-dimensional representations of the interior of the human body or of other imaged structures). Some diagnostic imaging protocols include one or more contrast scans, where a contrast agent is administered to the patient prior to the diagnostic imaging scan.

### SUMMARY

In one example, a method for a CT system comprises performing a CT scan of a patient injected with a contrast agent: reconstructing an image based on projection data acquired during the CT scan; adjusting a first contrast of a first anatomical region of the image based on a first set of display parameter settings of the CT system; adjusting a second contrast of a second anatomical region of the image based on a second set of display parameter settings of the CT system, the second anatomical region different from the first anatomical region, the second set of display parameter settings different from the first set of display parameter settings; displaying a contrast-optimized image on a display device of the CT system, the contrast-optimized image showing the first anatomical region of the image in the first contrast, and the second anatomical region of the image in the second contrast, the second contrast different from the first contrast.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various aspects of this disclosure may be better understood upon reading the following detailed description and upon reference to the drawings in which:

FIG. 1 shows a pictorial view of a CT imaging system, in accordance with one or more embodiments of the present disclosure;

FIG. 2A shows a block schematic diagram of an exemplary CT imaging system, in accordance with one or more embodiments of the present disclosure;

FIG. 2B shows an expanded view of an image visualization system of the exemplary CT imaging system of FIG. 2A, in accordance with one or more embodiments of the present disclosure;

FIG. 3 is a flowchart illustrating an exemplary method for post-processing a reconstructed image to create a customized visualization of anatomical regions of a subject of a CT scan, in accordance with one or more embodiments of the present disclosure;

FIG. 4 is a flowchart illustrating an exemplary method for generating a contrast-optimized image, in accordance with one or more embodiments of the present disclosure;

FIG. 5 is a reconstructed image showing anatomical reference points, in accordance with one or more embodiments of the present disclosure;

FIG. 6 is an exemplary non-contrast-optimized reconstructed CT image, as prior art;

FIG. 7 is a first exemplary contrast-optimized CT image, in accordance with one or more embodiments of the present disclosure;

FIG. 8 is a second exemplary contrast-optimized CT image of a patient;

FIG. 9 is a color map generated for the reconstructed grayscale CT image using material decomposition, as prior art;

FIG. 10 shows a first color overlay superimposed on the contrast-optimized CT image of FIG. 8, in accordance with one or more embodiments of the present disclosure;

FIG. 11 shows a second color overlay superimposed on the reconstructed grayscale CT image of FIG. 8, in accordance with one or more embodiments of the present disclosure; and

FIG. 12 shows an exemplary automated report generated by a CT system including one or more contrast-optimized CT images, in accordance with one or more embodiments of the present disclosure.

The drawings illustrate specific aspects of the described systems and methods. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems and methods.

### DETAILED DESCRIPTION

This description and embodiments of the subject matter disclosed herein relate to methods and systems for visualizing images reconstructed from scan data acquired via a computed tomography (CT) system. In CT imaging systems, an x-ray source typically emits a fan-shaped beam or a cone-shaped beam towards an object, such as a patient. X-rays emitted by the x-ray source are attenuated to varying degrees by the object prior to being detected by radiation detector elements arranged in one or more detector arrays. The x-ray source and the detector arrays are generally rotated about a gantry within an imaging plane and around the patient, and images are generated from projection data at a plurality of views at different view angles. The beam, after being attenuated by the patient, impinges upon the array of radiation detector elements. An intensity of the attenuated beam radiation received at the detector array is typically dependent upon the attenuation of the x-ray beam by the patient. Each detector element of a detector array produces a separate electrical signal indicative of the attenuated beam received by each detector element. The electrical signals are transmitted to a data processing system for analysis. The data processing system processes the electrical signals to facilitate generation of an image.

Conventional CT imaging systems utilize detectors that convert x-ray photon energy into current signals that are integrated over a time period, then measured and ultimately digitized. A draw back of such detectors is their inability to provide independent data or feedback as to the energy and incident flux rate of photons detected. That is, conventional CT detectors have a scintillator component and photodiode component wherein the scintillator component illuminates upon reception of x-ray photons and the photodiode detects illumination of the scintillator component and provides an integrated electrical current signal as a function of the intensity and energy of incident x-ray photons. These integrating detectors may not provide energy discriminatory data or otherwise count the number and/or measure the energy of photons actually received by a given detector element.

In contrast, an energy discriminating detector of a photon counting computed tomography (PCCT) system can provide photon counting and/or energy discriminating feedback with high spatial resolution. PCCT detectors can be caused to operate in an X-ray counting mode, and also in an energy measurement mode of each X-ray event. While a number of materials may be used in the construction of a direct conversion energy discriminating detector, semiconductors have been shown to be one preferred material. Typical materials for such use include Cadmium Zinc Telluride (CZT), Cadmium Telluride (CdTe) and Silicon (Si), which have a plurality of pixilated anodes attached thereto.

In x-ray projection systems and CT imaging modalities that do not utilize energy discrimination, the contrast between target objects and background objects is formed by differences in x-ray attenuation between target and background materials. Larger differences in x-ray attenuation translate to improved differentiation (e.g., higher contrast) of the target materials from the background materials. However, typically, images contain multiple materials and mixtures of materials that may yield similar contrasts in an x-ray projection or reconstructed CT image and make differentiation of the target objects difficult.

Conventional CT imaging can create a visualization of the density of the tissue and substances imaged in the subject. The density is derived as related to x-ray attenuation of the tissue and is encoded as a grey scale value in order to form an image. Density information is often used to segment regions of the images and associate those regions with certain biological tissues. For example, high attenuation is often associated with bone. By performing segmentation based on density information, it is possible to remove bone from the image so as to generate a soft-tissue image.

When performing CT imaging, intravascular contrast media or contrast agents, such as iodine agents, barium sulfate, etc., may be used to enhance image contrast. e.g., to “highlight” an organ of interest from surrounding tissue. Particularly, and with respect to an ROI, a contrast agent administered to a patient may have greater uptake in the ROI than in the other tissues. The contrast agent may be administered via a vein in an arm and/or other entry point using an injector with a single, bi or multi-phasic injection protocol or a catheter. Where a contrast agent bolus is administered, for a given location upstream from the injection site, contrast agent will initially be absent. The amount of contrast agent at that location will increase as the contrast agent distributes and enters the location (uptake) up to a peak amount (peak enhancement) and then decrease as the contrast agent exits the location (washout). In some cases, a CT scan may be configured to track peak enhancement during the scan, and CT scan timing may be optimized to view specific anatomy.

A quality of a reconstructed image may depend on a flow of an injected contrast agent through anatomies of the patient. The flow of an injected contrast agent through different anatomies may vary, and may differ based on contrast timing, contrast amounts, and physiologic factors (heart rate, respiratory rate, blood pressure, etc.). Contrast flow during CT exams has traditionally relied on anecdotal medical practice observation and expert interpretation to account for variables affecting flow rates and arrival times. Bolus tracking software may rely on additional monitoring scans and x-ray doses. Dual injection test bolus techniques rely on additional x-ray and contrast media doses. After a scan is performed, various reconstruction and visualization parameters may be adjusted for the different anatomies, based on contrast, and depending on the ROI. For example, a first set of reconstruction and/or visualization parameters may be used to highlight a first ROI in an image, or a second set of reconstruction and/or visualization parameters may be used to highlight a second ROI of the image.

Thus, an entire image volume may be customized for viewing a specific portion of a patient's anatomy, where the image volume may be less than ideal for viewing other portions of the patient's anatomy. As a result, reading multiple anatomies may entail generating multiple images. The multiple images may include monochromatic virtual images (MVI) (e.g., keV images) showing different anatomies in different contrasts, basis material decomposition (MD) images optimized to show features of specific anatomies, and/or other types of images. Generating the multiple images may increase a usage of the CT system and a workload of a radiologist operating the CT system and/or treating the patient. Increasing the usage of the CT system increases an amount of processing resources used for performing scans, increases a cost associated with treating the patient, and decreases an availability of the CT system for treating other patients.

As described in greater detail herein, the usage of the CT system and the processing resources consumed during scans may be reduced by performing an initial assessment of contrast timing and flow through different anatomical regions of a patient, and based on the initial assessment, applying different visualization schemes to the different anatomical regions of a single image, where each visualization is optimized for assessing a different anatomical region. The visualizations may increase a contrast between diseased tissues and non-diseased tissues of the different anatomical regions, and may include color maps (e.g., heat maps and/or probability maps) and/or color overlays based on spectral decomposition information. The different visualizations may be combined into a single visualization, such as a single 2D image, where each anatomical region (e.g., organ, bone, etc.) is displayed in high contrast, and where aspects of various anatomical regions may be highlighted or colorized to visualize specific information. Displaying the different visualizations of the different anatomical regions combined in the single 2D image may allow a radiologist to review multiple anatomies of the subject with data from one image volume, rather than different image volumes, where each image volume is reconstructed to optimize a contrast of a single anatomy. Consequently, the usage of the CT system may be minimized, and a time spent reading the multiple anatomies may be decreased, making a workflow of the radiologist more efficient while reducing a computational load on the CT system, thereby improving a functioning of the CT system overall.

Additionally, in some embodiments, comparisons between the visualizations including contrast optimization and/or color overlays and similar visualizations including contrast optimization and/or color overlays generated from prior studies may be automatically generated, for example, to show a progression of a disease detected in one or more anatomies of a patient over time. The comparisons may be generated automatically based on visualization techniques and parameters that are determined using various algorithms and/or deep learning methods. The comparisons may be summarized and outputted as a report that is automatically generated by the CT system, which a radiologist may view on a display device. By automatically determining the visualization techniques and parameters and generating the report, the workflow of the radiologist may be further simplified and a time of the radiologist spent generating and reviewing images may be further reduced.

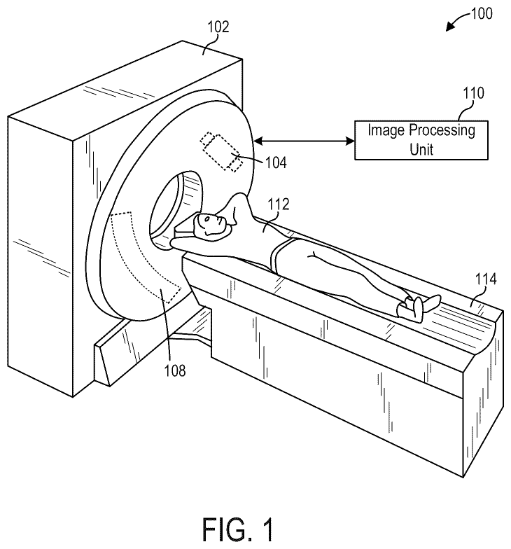

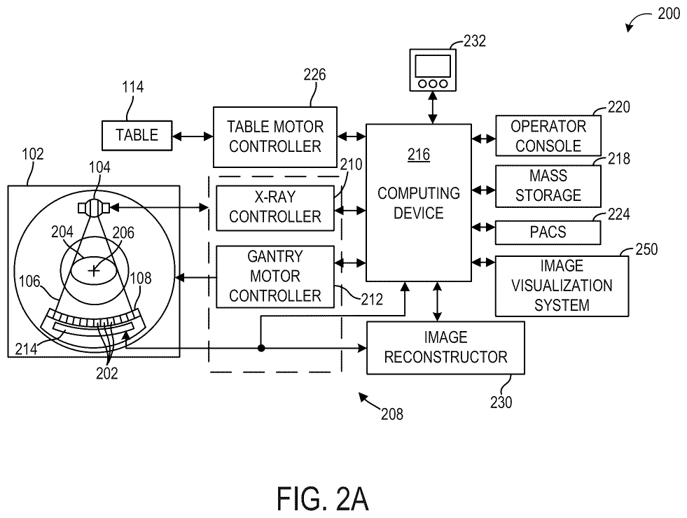

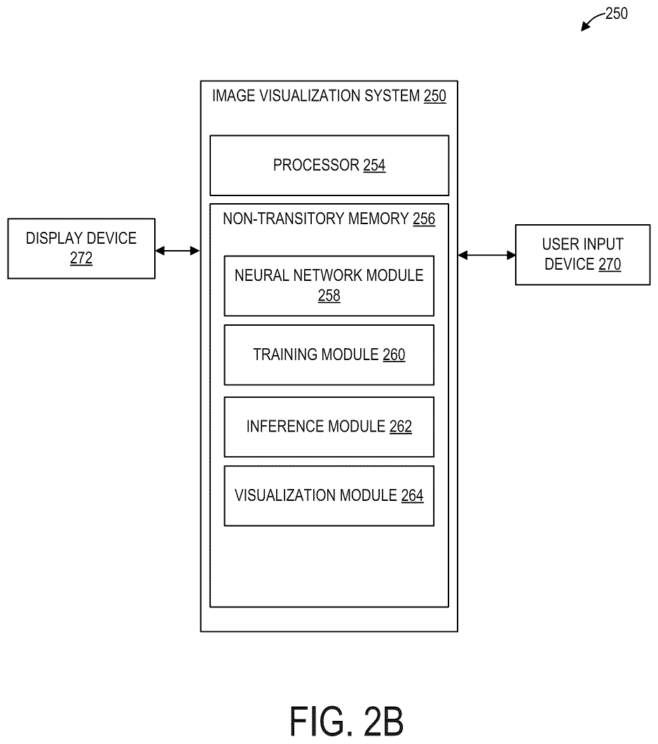

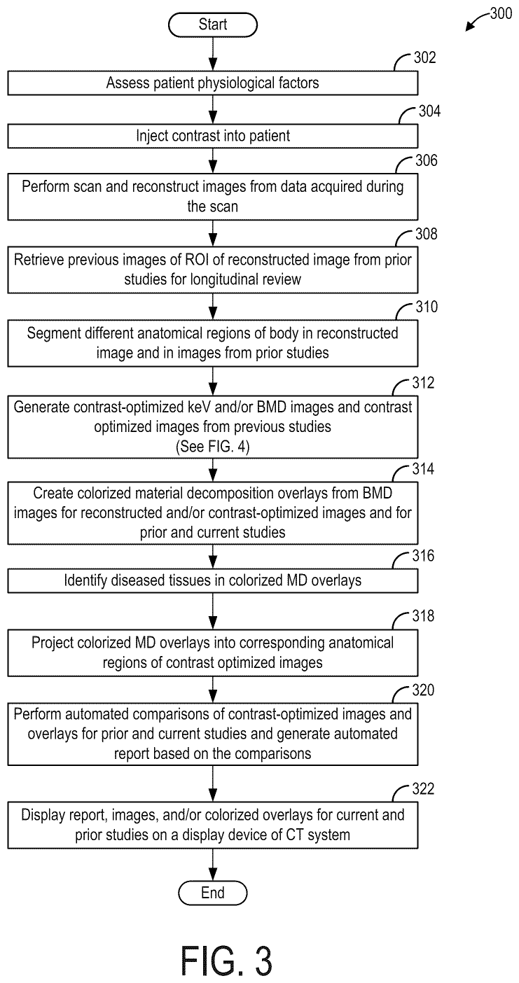

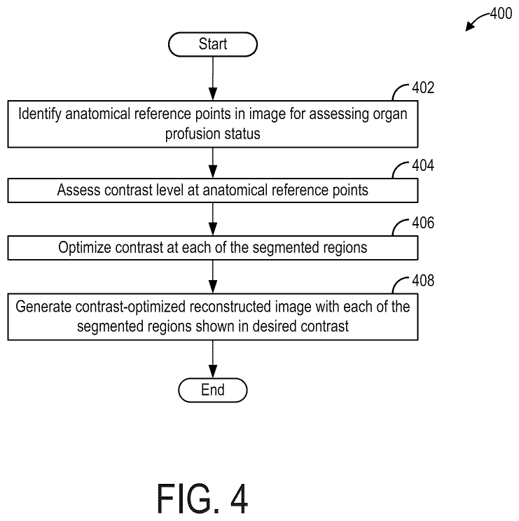

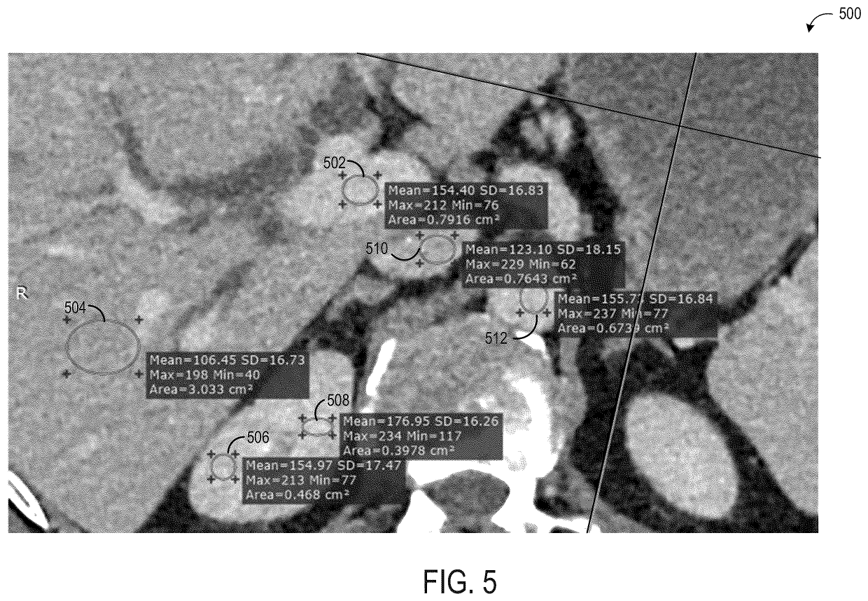

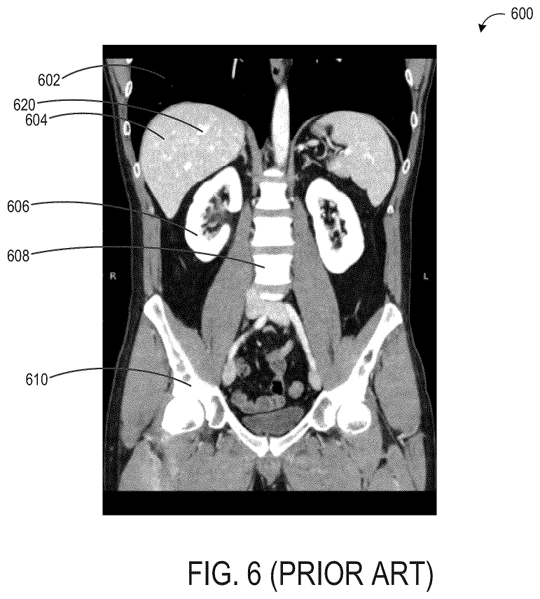

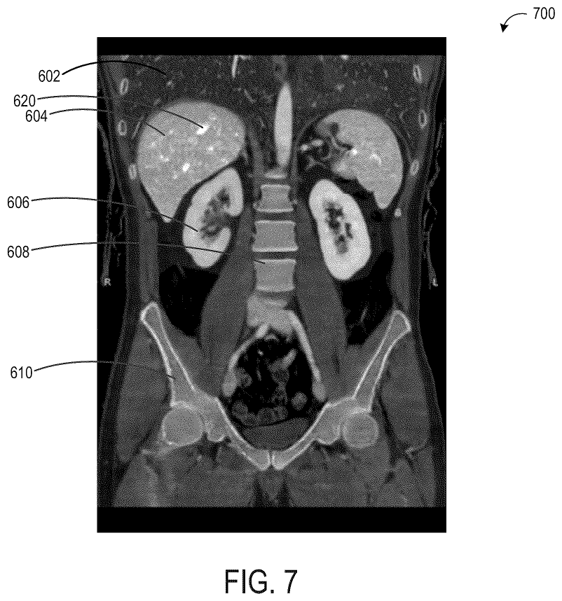

An exemplary CT system is provided in FIGS. 1 and 2A, and an image visualization system that may be included in or coupled to the exemplary CT system is shown in FIG. 2B. A scan may be performed using the CT system, and a grayscale (e.g., virtual monoenergetic) image may be reconstructed from projection data acquired during the scan. A visualization of the reconstructed image may be generated by following one or more steps of a method described in FIG. 3. The visualization may include a contrast-optimized visualization, where different anatomies are shown with different levels of contrast, which may be generated by following one or more steps of the method of FIG. 4. The contrast-optimized visualization may be generated based on an assessment of contrast uptake at various anatomical reference points, as shown in FIG. 5. A first exemplary 2D image of a contrast-optimized visualization is shown in FIG. 7, as compared with a second exemplary 2D image of a non-contrast-optimized visualization shown in FIG. 6.

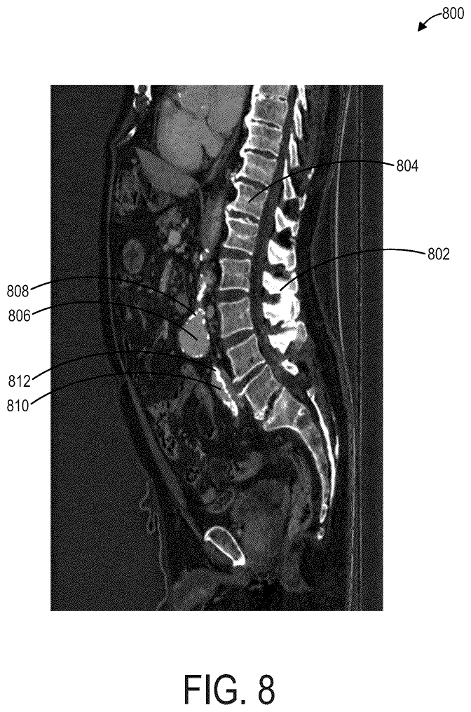

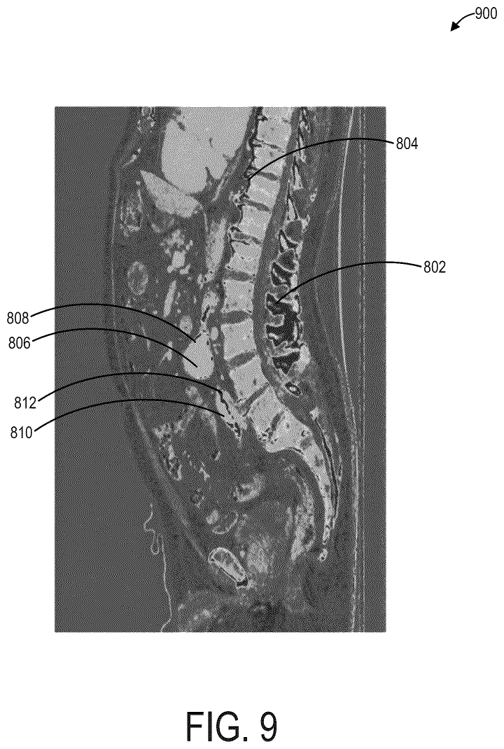

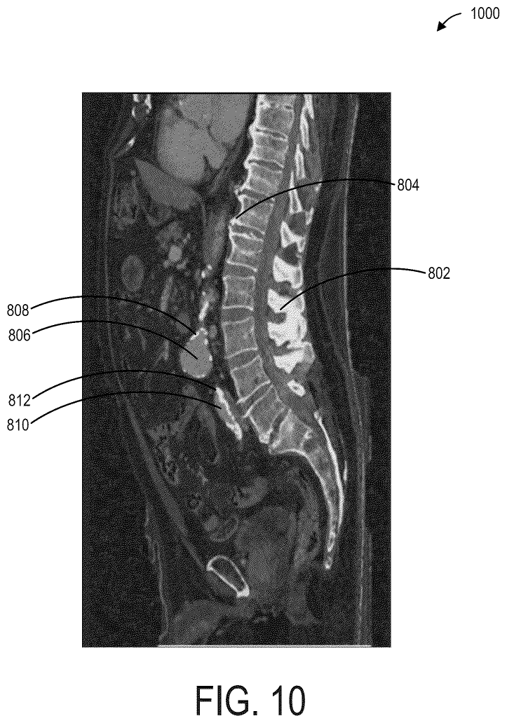

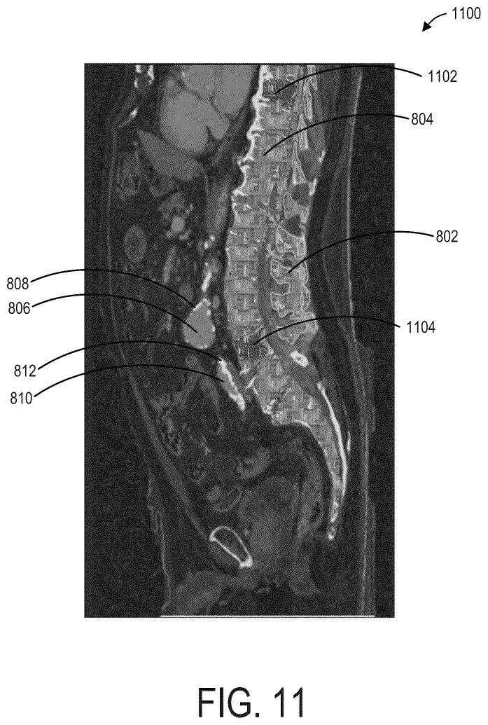

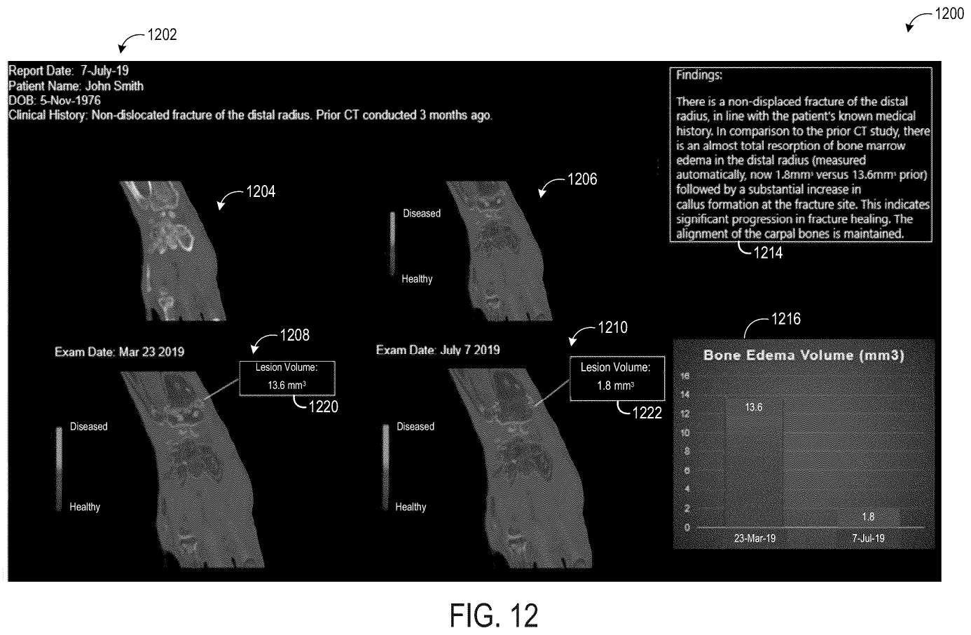

Additionally, colorized material decomposition overlays may be generated for different anatomical regions using MD. An exemplary color map generated using MD is shown in FIG. 9, in comparison to a contrast-optimized MVI shown in FIG. 8. A first example colorized material decomposition overlay superimposed on the contrast-optimized MVI of FIG. 8 is shown in FIG. 10, where the overlay removes bone tissue contrast data to highlight iodine uptake in soft tissues of a chest and abdomen of the patient. In addition to the first example colorized material decomposition overlay of FIG. 10, a second example colorized material decomposition overlay may be superimposed on the contrast-optimized MVI of FIG. 8, as shown in FIG. 11, where the second example colorized material decomposition overlay provides additional information specifically with respect to bone tissues. One or more visualizations such as those depicted in FIGS. 8-11 may be included in an automated report generated by the image visualization system, such as the automated report shown in FIG. 12.

Though a CT system is described by way of example, it should be understood that the present techniques may also be useful when applied to images acquired using other imaging modalities, such as x-ray imaging systems, magnetic resonance imaging (MRI) systems, positron emission tomography (PET) imaging systems, single-photon emission computed tomography (SPECT) imaging systems, ultrasound imaging systems, and combinations thereof (e.g., multi-modality imaging systems, such as PET/CT. PET/MR or SPECT/CT imaging systems). The present discussion of a CT imaging modality is provided merely as an example of one suitable imaging modality.

FIG. 1 illustrates an exemplary CT system 100 configured for CT imaging. CT system 100 is configured to image a subject 112 such as a patient, an inanimate object, one or more manufactured parts, and/or foreign objects such as dental implants, stents, and/or contrast agents present within the body. In one embodiment, the CT system 100 includes a gantry 102, which in turn, may further include at least one x-ray source 104 configured to project a beam of x-ray radiation 106 (see FIG. 2A) for use in imaging the subject 112 laying on a table 114. Specifically, the x-ray source 104 is configured to project the x-ray radiation beams 106 towards a detector array 108 positioned on the opposite side of the gantry 102. Although FIG. 1 depicts a single x-ray source 104, in certain embodiments, multiple x-ray sources and detectors may be employed to project a plurality of x-ray radiation beams for acquiring projection data at different energy levels corresponding to the patient. In some embodiments, the X-ray source 104 may enable dual-energy gemstone spectral imaging (GSI) by rapid peak kilovoltage (kVp) switching. In the embodiments described herein, the CT system 100 is a photon-counting computed tomography (PCCT) system, where the X-ray detector employed is a photon-counting detector which is capable of differentiating X-ray photons of different energies.

In certain embodiments, the CT system 100 further includes an image processing unit 110 configured to reconstruct images of a target volume of the subject 112 using an iterative or analytic image reconstruction method. For example, the image processing unit 110 may use an analytic image reconstruction approach such as filtered back projection (FBP) to reconstruct images of a target volume of the patient. As another example, the image processing unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. As described further herein, in some examples the image processing unit 110 may use an analytic image reconstruction approach, such as FBP, in addition to an iterative image reconstruction approach.

In some CT imaging system configurations, an x-ray source projects a cone-shaped x-ray radiation beam which is collimated to lie within an X-Y-Z plane of a Cartesian coordinate system and generally referred to as an “imaging plane,” The x-ray radiation beam passes through an object being imaged, such as the patient or subject. The x-ray radiation beam, after being attenuated by the object, impinges upon an array of detector elements. The intensity of the attenuated x-ray radiation beam received at the detector array is dependent upon the attenuation of an x-ray radiation beam by the object. Each detector element of the array produces a separate electrical signal that is a measurement of the x-ray beam attenuation at the detector location. The attenuation measurements from all the detector elements are acquired separately to produce a transmission profile.

In some CT systems, the x-ray source and the detector array are rotated with a gantry within the imaging plane and around the object to be imaged such that an angle at which the x-ray beam intersects the object constantly changes. A group of x-ray radiation attenuation measurements, e.g., projection data, from the detector array at one gantry angle is referred to as a “view,” A “scan” of the object includes a set of views made at different gantry angles, or view angles, during one revolution of the x-ray source and detector.

FIG. 2A illustrates an exemplary imaging system 200 similar to the CT system 100 of FIG. 1. In accordance with aspects of the present disclosure, the imaging system 200 is configured for imaging a subject 204 (e.g., the subject 112 of FIG. 1). In one embodiment, the imaging system 200 includes the detector array 108 (see FIG. 1). The detector array 108 further includes a plurality of detector elements 202 that together sense the x-ray radiation beam 106 that pass through the subject 204 (such as a patient) to acquire corresponding projection data. In some embodiments, the detector array 108 may be fabricated in a multi-slice configuration including the plurality of rows of cells or detector elements 202, where one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the x-ray source 104 and the detector array 108 rotate, the detector array 108 collects data of the attenuated x-ray beams. The data collected by the detector array 108 undergoes pre-processing and calibration to condition the data to represent the line integrals of the attenuation coefficients of the scanned subject 204. The processed data are commonly called projections. In some examples, the individual detectors or detector elements 202 of the detector array 108 may include photon-counting detectors which register the interactions of individual photons into one or more energy bins.

The acquired sets of projection data may be used for basis material decomposition (MD). During MD, the measured projections are converted to a set of material-density projections. The material-density projections may be reconstructed to form a pair or a set of material-density map or image of each respective basis material, such as bone, soft tissue, and/or contrast agent maps. The density maps or images may be, in turn, associated to form a 3D volumetric image of the basis material, for example, bone, soft tissue, and/or contrast agent, in the imaged volume. The material-density projections may also be used to generate colorized overlays (also referred to herein as color overlays) for reconstructed images.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In some embodiments, the density maps may be used to generate color overlays for a reconstructed image, where the color overlays may highlight portions of the reconstructed image or display additional information relevant to portions of the reconstructed image. Examples of such color overlays are shown in FIGS. 10 and 11, which may be generated in accordance with the method of FIG. 3.

In one embodiment, the imaging system 200 includes a control mechanism 208 to control movement of the components such as rotation of the gantry 102 and the operation of the x-ray source 104. In certain embodiments, the control mechanism 208 further includes an x-ray controller 210 configured to provide power and timing signals to the x-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate analog data from a subset of the detector elements 202 into so-called macro-detectors, as described further herein. The data sampled and digitized by the DAS 214 is transmitted to a computer or computing device 216. It is noted that the computing device 216 may be the same or similar to image processing unit 110, in at least one example. In one example, the computing device 216 stores the data in a storage device or mass storage 218. The storage device 218, for example, may be any type of non-transitory memory and may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands, scanning parameters, and/or display parameters via an operator console 220 operatively coupled to the computing device 216. The operator console 220 may include a keyboard (not shown) or a touchscreen to allow the operator to specify the commands, scanning parameters, and/or display parameters.

Although FIG. 2A illustrates one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized x-ray data to perform high-speed reconstruction. Although FIG. 2A illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a “cloud” network cluster for the image reconstructor 230).

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device (e.g., operator console) 232 communicatively coupled to the computing device 216 and/or the image reconstructor 230). In some embodiments, the reconstructed images may be transmitted from the computing device 216 or the image reconstructor 230 to the storage device 218 for short-term or long-term storage.

Imaging system 200 may include, or be coupled to, an image visualization system 250. Image visualization system 250 may perform post-processing on the reconstructed images for purposes of visualization of features of the reconstructed images. During the post-processing of an image, various visualization parameters may be adjusted to increase a quality or readability of portions of interest of the image. For example, a contrast of the image may be increased. Additionally, as described in greater detail below, a contrast of different portions of the image may be individually adjusted. By individually adjusting the contrast of the different portions, a radiologist may be able to read multiple anatomies in a single visualization of the image.

FIG. 2B shows an expanded view of image visualization system 250 of imaging system 200, in accordance with an embodiment. In some embodiments, at least a portion of image visualization system 250 is disposed at a device (e.g., edge device, server, etc.) communicably coupled to imaging system 200 via wired and/or wireless connections. In some embodiments, at least a portion of image visualization system 250 is disposed at a separate device (e.g., a workstation) which can receive images from imaging system 200 or from a storage device such as PACS 224, which stores the images/data generated by imaging system 200.

Image visualization system 250 includes a processor 254 configured to execute machine readable instructions stored in non-transitory memory 256. Processor 254 may be single core or multi-core, and the programs executed thereon may be configured for parallel or distributed processing. In some embodiments, the processor 254 may optionally include individual components that are distributed throughout two or more devices, which may be remotely located and/or configured for coordinated processing. In some embodiments, one or more aspects of the processor 254 may be virtualized and executed by remotely-accessible networked computing devices configured in a cloud computing configuration.

Non-transitory memory 256 may store a neural network module 258, a training module 260, an inference module 262, and a visualization module 264. Neural network module 258 may include one or more deep learning networks and instructions for implementing the deep learning networks. For example, in some embodiments, neural network module 258 may include instructions for training a neural network to identify diseased tissues in a selected anatomy of a reconstructed image. Neural network module 258 may include one or more trained and/or untrained neural networks and may further include various data, or metadata pertaining to the one or more neural networks stored therein.

Training module 260 may comprise instructions for training one or more of the neural networks implementing a deep learning model stored in neural network module 258. In particular, training module 260) may include instructions that, when executed by the processor 254, cause image visualization system 250) to conduct one or more of the steps of method 1300 for training the one or more neural networks. In some embodiments, training module 260 includes instructions for implementing one or more gradient descent algorithms, applying one or more loss functions, and/or training routines, for use in adjusting parameters of the one or more neural networks of neural network module 258.

Non-transitory memory 256 also stores an inference module 262 that comprises instructions for deploying a trained deep learning model of neural network module 258. For example, inference module 262 may store a set of instructions that when executed by processor 254, cause image visualization system 250 to deploy a neural network model to identify diseased tissues in a selected anatomy of an image, or differentiate between harder and softer tissues, as described in greater detail below. The neural network model may be deployed by visualization module 264, which may include instructions that when executed by processor 254, may generate a custom visualization of the image including a contrast-optimized visualization and/or one or more colorized material decomposition overlays. The custom visualization may be generated by performing one or more steps of the method of FIG. 3 described below.

In some embodiments, the non-transitory memory 256 may include components disposed at two or more devices, which may be remotely located and/or configured for coordinated processing. In some embodiments, one or more aspects of the non-transitory memory 256 may include remotely-accessible networked storage devices configured in a cloud computing configuration.

Image visualization system 250 may be operably/communicatively coupled to a user input device 270 and a display device 272. User input device 270 may comprise one or more of a touchscreen, a keyboard, a mouse, a trackpad, a motion sensing camera, or other device configured to enable a user to interact with and manipulate data within image visualization system 250. Display device 272 may include one or more display devices utilizing virtually any type of technology. In some embodiments, display device 272 may comprise a computer monitor, and may display medical images. Display device 272 may be combined with processor 254, non-transitory memory 256, and/or user input device 270 in a shared enclosure, or may be peripheral display devices and may comprise a monitor, touchscreen, projector, or other display device known in the art, which may enable a user to view medical images produced by a medical imaging system, and/or interact with various data stored in non-transitory memory 256. In some embodiments, user input device 270) and display device 272 may be incorporated into operator console 232 of FIG. 2A.

It should be understood that image visualization system 250 shown in FIG. 1 is for illustration, not for limitation. Another appropriate image visualization system may include more, fewer, or different components.

FIG. 3 shows a flowchart illustrating an exemplary method 300 for generating a contrast-optimized image from scan data acquired via a CT imaging system, such as CT imaging systems 100 and 200 described above. Method 300 may be stored as instructions in a non-transitory memory and executed by one or more processors of a computing device of the CT imaging system, such as computing device 216 of imaging system 200 of FIG. 2A.

Method 300 begins at 302, where method 300 includes assessing physiological factors of the patient. The physiological factors the patient may include, for example, a heart rate measured via a CT scanner of the CT imaging system: an oxygen saturation of blood of the patient: a respiratory rate of the patient: a blood pressure of the patient; and/or other physiological factors. The physiological factors may determine how the contrast agent administered to the patient flows through the body and the uptake in various anatomies of the patient.

At 304, method 300 includes injecting an amount of contrast agent into the patient, based on the assessed physiological factors. The contrast agent may be a substance, such as an iodine- or barium-based substance, that attenuates x-ray beams generated by the CT imaging system. By attenuating the x-ray beams, the contrast agent may increase a radiodensity of tissues and/or anatomical structures of a body of the patient, thereby increasing a contrast between different types of the tissues and/or anatomical structures. The increased contrast may make it easier to view the tissues and/or anatomical structures on a display device of the CT imaging system. After injecting the contrast agent into the patient, the patient may rest for a waiting period, during which the contrast may flow throughout an ROI and be taken up by tissues of the ROI (e.g., bone, soft tissues, organs, etc.).

A length of the waiting period may depend on the physiological factors of the patient. For example, a first patient may have a first set of physiological factors, where as a result of having the first set of physiological factors, the contrast agent injected into the body of the first patient may be taken up by an organ of the first patient at a first uptake rate. A second patient may have a second set of physiological factors, where as a result of having the second set of physiological factors, the contrast agent injected into the body of the second patient may be taken up by the organ of the second patient at a second uptake rate. The second uptake rate may be lower than the first uptake rate, where less contrast agent is taken up by the organ of the second patient than the organ of the first patient. As a result of less contrast agent uptake by the organ of the second patient, an image generated from a scan of the second patient may show the organ with less contrast than an image of the organ of the first patient. Thus, the physiological factors may be used to estimate a contrast uptake rate of the patient, and the waiting period may be based on the estimated contrast uptake rate and an amount of contrast desired in a resulting image.

At 306, method 300 includes performing a scan using the CT system to obtain detector data, and reconstructing one or more images (e.g., image volumes) from data acquired during the scan. The one or more images may include MVIs. MD images, and/or other types of images. Performing the scan may include selecting a protocol to apply during the scan, which may be selected based on a ROI of the patient and/or various other factors. The image may be reconstructed using reconstruction parameters determined by the protocol and/or an operator of the CT system. Additionally, in various examples, the reconstruction parameters may be selected based on a scout scan or scanogram performed on the patient using the CT imaging system prior to performing the scan. The scout scan may be an initial scan performed with a smaller radiation dose than a typical imaging scan, and may be performed without rotating a gantry of the CT imaging system (e.g., gantry 102 of FIG. 1).

In various embodiments, the CT system may be a PCCT system, where the detector data includes bin counts. The detector data may include, for each pixel of the photon-counting detector (e.g., for each detector element 202), photon counts partitioned into a plurality of energy bins based on an energy imparted by each photon on the photon-counting detector, which is referred to as bin counts herein. During the scan, the X-ray source (e.g., X-ray source 104 of FIGS. 1 and 2) of the CT imaging system may be controlled to emit X-rays according to a scan prescription set forth by the selected scan protocol (e.g., with a set X-ray source or

X-ray tube current and voltage). Detector data may be obtained from the detector array (e.g., detector array 108 of FIGS. 1 and 2) as the X-ray source and detector array rotate around the patient, resulting in a plurality of views of detector data being obtained. For each view, a photon count for each energy bin for each pixel or detector element of the detector array is generated (e.g., during readout of the detector array by DAS 214). For example, in detector configurations with eight energy bins, eight photon counts may be generated for each pixel or detector element of the detector array and for each view.

In this way, the output of the detector array may be referred to as the bin counts, as the photon counts are partitioned into energy bins based on the energy of each photon that impinges on the detector array. The number of energy bins may be based on the configuration of the detector. For example, silicon detectors may be configured to differentiate photon energy into 8 energy bins, while cadmium telluride detectors may be configured to differentiate photon energy into 5 bins. The energy thresholds that define the energy bins may be determined during a calibration phase and/or may be based on the specific scan protocol. For example, the energy thresholds may be determined to optimize material basis decomposition and/or to maximize detected spectral information for a given incident spectrum emitted by the X-ray source. In a non-limiting example, the energy bin thresholds may be 4, 14, 30, 37, 47, 58, 67, and 79 keV for an 8 bin detector, or 10, 34, 50, 62, and 76 keV for a 5 bin detector.

At 308, method 300 includes retrieving previous images of a ROI of the reconstructed image from prior studies, for a longitudinal review. The prior studies may be stored in a PACS of the CT system, such as PACS 224 of FIG. 2A. The previous images may include various types of images that share anatomical structures, features, or other aspects with the reconstructed image, such as monoenergetic virtual CT images. MD images. MRI images. X-Ray images, or a different kind of image. The previous images may include colorized images or overlays, such as heat maps or probability maps generated from spectral decomposition or other techniques that highlight specific data in the previous images. Standard image registration techniques may be used for comparison across different studies.

The previous images may be selected from various prior studies in accordance with one or more algorithms based on series data and/or other metadata or header data associated with the images. For example, if the ROI includes bone tissues, water images may be selected for bone marrow and calcium/hydroxyapatite images may be selected for dense bone. A relevant series of the prior studies may be selected based on the series data. Images specific to a disease of the patient and/or images of specific anatomies of the patient may be selected for segmentation.

In some embodiments, the one or more algorithms may be selected by a rules-based system that selects relevant images from the prior studies based on logic rules and lookup tables. For example, a set of parameters may be identified in the current scan, and the set of parameters may be matched to the prior studies of the same patient stored on the PACS. Additionally or alternatively, a machine learning (ML) or deep learning (DL) model including one or more neural networks may be trained to select suitable images from the prior studies.

At 310, method 300 includes performing a segmentation process to segment different anatomical regions of the patient's body in the reconstructed image. For example, a segmentation of an abdomen of the patient may result in identifying a first portion of the image (e.g., a set of voxels of the image) corresponding to a liver of the patient: a second portion of the image corresponding to a kidney of the patient: a third portion of the image corresponding to a pelvis of the patient; and so on. Segmenting of the different anatomical regions may be performed using various techniques or methodologies known in the art. For example, in one embodiment, the segmentation may be performed by a segmentation model comprising a neural network trained to detect boundaries of different anatomical regions. Detection of the boundaries may facilitate a post-reconstruction optimization of a contrast of the different anatomical regions, as described below.

In various embodiments, the same segmentation process may be performed on the retrieved images from the prior studies. It should be appreciated that the processes performed in steps 310-318 on the reconstructed image may also be applied to the retrieved images. Further, the processes described below may be performed on the reconstructed image and on the retrieved images using a same set of parameters, such that a first visualization generated from the reconstructed image and a second visualization (or additional visualizations) generated from a retrieved image may be compared under conditions as close to equal as possible. Comparing visualizations generated from scan data acquired at different times is described in greater detail below in reference to step 320.

At 312, method 300 includes generating one or more contrast-optimized keV and/or MD images from the images reconstructed at 308, and contrast-optimized images from prior studies. The contrast-optimized images may show anatomical regions of the patient in different desired levels of contrast. For example, in a contrast-optimized image, display or visualization parameters of the CT system may be adjusted such that a first set of display parameter settings are selected to show a first anatomical region in a first contrast, and a second set of display parameter settings are selected to show a second anatomical region in a second contrast, where the second set of display parameter settings is different from the first set of display parameter settings, and the second contrast is different from the first contrast. Generation of the one or more contrast-optimized images is described in greater detail below in reference to FIG. 4.

At 314, method 300 includes generating one or more colorized overlays from material decomposition data acquired via the CT imaging system for the prior studies and the current study. The material decomposition overlays may be created using MD. MD is based on the concept that the x-ray attenuation of any given material in the energy range can be represented by a linear combination of a density mixture of other known materials, referred to as basis materials. Using MD, a plurality of reconstructed images may be obtained, each image representing the equivalent density of one of the basis materials. Since density is independent of x-ray photon energy, these images may be relatively free of beam hardening artifacts. The basis materials may be chosen to target a material of interest, thus enhancing the image contrast. Overlays may then be created based on the images reconstructed using MD, where one or more relevant anatomical features are colorized based on the MD images, and other portions of the overlays may be transparent. The overlays may then be superimposed on 2-D images including the relevant anatomical features, where the colorized portions may show additional information not included in the 2-D images.

In other words, a plurality of material decomposition overlays may be generated using MD for a respective plurality of basis materials. Each color overlay of the plurality of material decomposition overlays may include anatomical regions having a 1:1 correspondence with anatomical regions of the contrast-optimized image, with respect to size and positioning of the anatomical regions with respect to other anatomical regions and/or structures of the patent. Each material decomposition overlay may show different anatomical regions of the patient's body in different colors. For example, a first material decomposition overlay may highlight a first anatomy of the patient using a first set of colors to differentiate between tissues of the first anatomy: a second material decomposition overlay may highlight a second anatomy of the patient using a second set of colors to differentiate between tissues of the second anatomy: a third material decomposition overlay may highlight a third anatomy of the patient using a third set of colors to differentiate between tissues of the third anatomy; and so on.

Further, different colors may be applied to different portions of an anatomical region. For example, a first portion of an anatomical region with a first set of characteristics may be displayed in a first color, and a second portion of the anatomical region with a second set of characteristics may be displayed in a second color, where a difference between the first color and the second color may highlight or distinguish tissues from having the first set of characteristics from tissues having the second set of characteristics. For example, diseased tissues may be displayed in the first color, and healthy tissues may be displayed in the second color. Additionally or alternatively, harder bone cortex tissues may be shown in one color, and softer bone marrow tissues may be shown in a different color. Distinguishing between diseased and healthy tissues and between harder and softer bone tissues may be accomplished using one or more DL models, as described in greater detail below. In this way, a first set of colors may be applied to a first anatomical region: a second set of colors may be applied to a second anatomical region, where the second set of colors is different from the first set of colors: a third set of colors may be applied to a third anatomical region, where the third set of colors is different from the first and second sets of colors; and so on.

Additionally, after the material decomposition overlays are generated, contrast-optimized versions of the material decomposition overlays may be generated by following a process such as the method of FIG. 4. The contrast-optimized versions may more clearly visualize different anatomical regions of the patient's body where each contrast-optimized version may show a relevant anatomical region in a different contrast. Examples of material decomposition overlays are described in greater detail below, in reference to FIGS. 8, 9, 10, and 11.

It should be appreciated that the processing of the reconstructed image described in method 300 to generate the contrast-optimized image and/or the colorized overlays may also be applied to previous reconstructed images of prior studies, for purposes of comparison. For example, a prior study matching the patient and relevant anatomy may be retrieved from a PACS, based on scan parameters, protocols, etc. A previous reconstructed image may be extracted from the retrieved prior study, and a second contrast-optimized image may be generated from the previous reconstructed image by following one or more steps of method 300. A second colorized overlay may then be generated and superimposed on the second contrast-optimized image, where the second colorized overlay is generated using a same method and same set of parameters as a first colorized overlay superimposed on the contrast-optimized image of the current scan. The second contrast-optimized image including the second colorized overlay may then be compared to the contrast-optimized image including the first colorized overlay.

Performing a similar process on both of the current reconstructed image and the previous reconstructed image may facilitate a more efficient visual comparison by a radiologist, and may provide for an automated comparison of features between the second contrast-optimized image including the second colorized overlay and the contrast-optimized image including the first colorized overlay. For example, a second size of a lesion in the second contrast-optimized image including the second colorized overlay may be calculated and compared with a first size of the lesion in the contrast-optimized image including the first colorized overlay. A difference between the first size and the second size may indicate a progression of a disease of the patient, as described in greater detail below.

At 316, method 300 includes identifying diseased tissues in the colorized MD overlays and/or contrast-optimized images. The (contrast-optimized) material decomposition overlays may be used by a radiologist to detect diseased tissues, based on characteristics of the anatomical regions highlighted by the colors and/or contrast optimization. Additionally, one or more models may be used to automatically detect and/or segment diseased tissues of the anatomical regions, and indicate the detected diseased tissues in the material decomposition overlays via highlighting, color, shading, outlining, etc. For example, in some embodiments, the diseased tissues may be identified by a tissue assessment DL model, such as a convolutional neural network (CNN). The tissue assessment DL model may be trained to identify the diseased tissues using labeled, ground truth monochromatic, material density, and/or MR images. In other embodiments, the diseased tissues may be identified using a different type of model or technique.

At 318, method 300 includes projecting the colorized material decomposition overlays onto corresponding anatomical regions of the contrast optimized images generated at 312. A single overlay may be projected (e.g., superimposed) on a single contrast image, or a plurality of overlays may be projected on a single contrast image. For example, a first color overlay may be superimposed on a contrast-optimized image that applies a first color to a first segmented anatomical region of the contrast-optimized image. A second color overlay may be additionally superimposed on the contrast-optimized image that applies a second color to a second segmented anatomical region of the contrast-optimized image. The first color overlay may selectively apply the first color to the first segmented anatomical region, and apply no color to the second segmented anatomical region and other anatomical regions. The second color overlay may selectively apply the second color to the second segmented anatomical region, and apply no color to the first segmented anatomical region and other anatomical regions. In this way, each color overlay may increase an amount of information (e.g., spectral decomposition information) presented to a viewer with respect to a particular anatomical region, without altering the display and the amount of information presented with respect to other anatomical regions.

Further, a color overlay may selectively apply a first color to a first portion of an anatomical region, and a second color to a second portion of the anatomical region. For example, the color overlay may display diseased tissues of the anatomical region in a first color, and display healthy tissues of the anatomical region in a second color, where application of the first color and the second color highlight the diseased tissues. The diseased tissues may be identified based on an amount of injected contrast agent uptake by the diseased tissues in comparison to a second amount of the injected contrast agent uptake by the healthy tissues. In some embodiments, an ML model may be used to identify the diseased tissues. For example, the ML model may take as input an image of the anatomical region, and may output a segmentation of diseased tissues in the anatomical region. In other embodiments, the diseased tissues may be identified in a different manner. In this way, by superimposing (e.g., stacking) a plurality of color overlays on a reconstructed image, a visualization may be generated that shows a plurality of anatomical regions in a desired (e.g., ideal) contrast, while additionally highlighting areas of diseased tissue within one or more anatomical regions using color.

At 320, method 300 includes performing automated comparisons of contrast-optimized images and composite color overlays for the prior and current studies and generating an automated report based on the automated comparisons of the current and prior studies. The automated report may show one or more trends in data displayed in the visualizations of the current and prior studies. Specifically, differences between a first contrast-optimized image and a second (previous) contrast-optimized image may be measured, and a trend may be inferred based on one or more measured differences.

As one example, a progression of a disease in one or more of the segmented regions may be determined by comparing prior visualizations with current visualizations. For example, the automated report may include a first contrast-optimized image from a current scan performed on the patient, and a second contrast-optimized image from a prior scan performed on the patient. The second contrast-optimized image may be stored in a PACS, or the second contrast-optimized image may be generated from a previous reconstructed image stored in the PACS by performing the same steps used to generate the first contrast-optimized image (e.g., steps 310-318). In other words, generating the second contrast-optimized image may include performing an identical segmentation process on the previous reconstructed image as performed on the current reconstructed image: identifying the same anatomical reference points in the previous reconstructed image as used in the current reconstructed image: assessing a contrast level of the previous reconstructed image at the same anatomical reference points; selectively adjusting the same sets of display parameters for anatomical regions of the previous reconstructed image using the same settings that were used for the current reconstructed image; identifying healthy and diseased tissues in the previous reconstructed image using the same techniques, tools, or DL models used to identify healthy and diseased tissues in the current reconstructed image; and generating and superimposing colorized overlays on the previous reconstructed image using the same parameters, techniques, and models used to generate and superimpose colorized overlays on the current reconstructed image. In this way, the second contrast-optimized image and the first contrast-optimized image may have a high degree of similarity, where notable differences between the second contrast-optimized image and the first contrast-optimized image may be based on a progression of a disease of the patient.

Both of the first contrast-optimized image and the second contrast-optimized image may show an area of diseased tissue of the patient. A first segmentation may be performed on the area of diseased tissue in the first contrast-optimized image, and a second segmentation may be performed on the area of diseased tissue in the second contrast-optimized image. For example, the area of diseased tissue may be segmented by an ML model trained to detect areas of high contrast with certain properties in a reconstructed image. Based on the segmentation, the image visualization system may calculate a first size of the area of diseased tissue in the first contrast-optimized image, and a second size of the area of diseased tissue in the second contrast-optimized image. The image visualization system may determine that the first size is smaller than the second size, whereby the image visualization system may determine that the size of the diseased area is decreasing over time. Additionally, based on a difference between the first size and the second size, the image visualization system may estimate a progression of the disease. The progression of the disease may be described and presented textually in the automated report.

A radiologist reviewing the report may also view the first contrast-optimized image and the second contrast-optimized image side-by-side, where the progression of the disease and the relative size of the area of diseased tissue may be directly compared between the first contrast-optimized image and the second contrast-optimized image. In some embodiments, the area of diseased tissue and/or other aspects of anatomical features of the patient may be labeled or indicated in either or both of the first contrast-optimized image and the second contrast-optimized image.

The automated report may include various information, analysis, diagnoses, visualizations, comparisons, trends over time, graphs, etc., which may be differ and/or be presented in a customized manner depending on availability of data, anatomical region, results of the analysis, and/or other factors. For example, the automated report may texturally summarize or indicate findings of the image visualization system. An exemplary automated report is described in greater detail below in reference to FIG. 12.

At 322, method 300 includes displaying the automated report and/or contrast-optimized image optionally including one or more colorized overlays, along with other contrast-optimized images and color overlays of the prior studies, on a display device of the CT imaging system/image visualization system (e.g., display device 272 of FIG. 2B). The contrast-optimized image may show relevant aspects of anatomical regions, such as diseased tissues, in a higher contrast and/or in a manner that makes it easier to quickly and efficiently diagnose the patient. In some examples, a user such as a radiologist may compare the contrast-optimized image with the initial reconstructed image.

Turning now to FIG. 4, an exemplary method 400 is shown for generating a contrast-optimized image from an image reconstructed from scan data acquired from a patient using a CT system, such as CT imaging systems 100 and 200 described above. The contrast-optimized image may show various anatomical regions of the patient in higher contrast or increased quality with respect to the reconstructed image. By viewing the contrast-optimized image, a radiologist may or quickly and efficiently identify abnormalities, such as areas of diseased tissue in the anatomical regions. Additionally, the contrast-optimized image may be included in a report, such as the automated report described above in reference to FIG. 3. Method 400 may be stored as instructions in a non-transitory memory and executed by one or more processors of a computing device of the CT imaging system, such as computing device 216 of imaging system 200 of FIG. 2A.

At 402, method 400 includes identifying a plurality of anatomical reference points in the reconstructed image, for assessing an organ perfusion status of a contrast agent injected into the patient during the scan. In some embodiments, a first neural network may be used for segmentation of the target anatomy, and a secondary neural network may be used for classification of the contrast phase in the image. In other words, a timing and flow of the contrast agent through the patient's body is assessed to determine a degree to which the contrast agent has been taken up into different anatomical regions of the patient's body. The contrast agent may include glucose, whereby the contrast agent uptake may be greater by cells with higher metabolic activity, such as cancer cells. Areas where the contrast agent uptake has been greater may appear brighter than areas where the contrast agent uptake has been to a lesser degree.

At 404, method 400 includes assessing a contrast level at each anatomical reference point of the plurality of anatomical reference points. The contrast level may be a measurement of an opacity of elements of the reconstructed image at the anatomical reference point, based on the absorption of the contrast agent at the anatomical reference point indicated in Hounsfield units. In some embodiments, the contrast level may be assessed using histogram data of the image at the anatomical reference point. For example, a first amount of contrast may be taken up at a first anatomical reference point of a first segmented anatomical region, and a second, lesser amount of contrast may be taken up at a second anatomical reference point of a second segmented anatomical region. As a result of a greater uptake of contrast, the first segmented anatomical region may appear brighter than desired when the reconstructed image is displayed on a display device. As a result of the lesser contrast uptake, the second segmented anatomical region may appear darker than desired when the reconstructed image is displayed on a display device. Thus, achieving an ideal contrast at both of the first anatomical region and the second anatomical region may not be possible by making a global adjustment to display parameters of the CT system, where a first global adjustment to decrease the brightness of the first anatomical region may further darken the second anatomical region, and a second global adjustment to increase the brightness of the second anatomical region may further brighten the second anatomical region.

Referring briefly to FIG. 5, a portion of a reconstructed image 500 is shown, where a plurality of anatomical reference points 502, 504, 506, 508, 510, and 512 have been identified. Each of anatomical reference points 502, 504, 506, 508, 510, and 512 may have a different size/area. A contrast level may be calculated for each of the plurality of anatomical reference points 502, 504, 506, 508, 510, and 512.

For example, a first contrast level may be calculated for anatomical reference point 502, corresponding to a portal vein of the patient. A second contrast level may be calculated for anatomical reference point 504, corresponding to a liver of the patient. A third contrast level may be calculated for anatomical reference point 506, corresponding to a kidney cortex of the patient. A fourth contrast level may be calculated for anatomical reference point 508, corresponding to a kidney medulla of the patient. A fifth contrast level may be calculated for anatomical reference point 510, corresponding to an inferior vena cava of the patient. A sixth contrast level may be calculated for anatomical reference point 512, corresponding to an aorta of the patient. Each of the first, second, third, fourth, and fifth contrast levels may be different.

Returning to method 400, at 406, method 400 includes optimizing a contrast of the reconstructed image at each of the segmented regions, based on the protocol and the contrast level assessed at each anatomical reference point. The optimization of the contrast at each of the segmented regions may be performed automatically. Specifically, a set of one or more algorithms may be applied to the reconstructed image volume to determine how the contrast level of the image within the segmented regions may be adjusted. Different processes may be performed on different segmented regions to adjust the contrast level of the image within the different segmented regions.

For example, adjusting the contrast level of bone tissues may include adjusting settings of a first set of one or more display (e.g., post-reconstruction) parameters of the CT system, while adjusting the contrast level of different types of softer tissues may include adjusting settings of different sets of one or more display parameters (also referred to herein as visualization parameters) of the CT system. Thus, the processes and/or algorithms performed to optimize the contrast at a specific segmented region may be customized for the specific segmented region, and may not be performed to optimize the contrast at other segmented regions.

The visualization parameters may include a window width (WW) and/or a window level (WL) setting applied to each of the segmented regions. The WW and the WL settings may be based on an expected range of values, in Hounsfield units, generated for each voxel during image reconstruction. Adjusting the WW and WL settings may change how tissue attenuation measurements are translated into a grayscale image, and optimal WW and WL settings may differ for different types of tissues. For example, a wider WW may result in a higher contrast when visualizing bone tissues than a narrower WW, while the narrower WW may result in a higher contrast when visualizing soft tissues than the wider WW. The WL is a middle value of the range of values included in the WW. Adjusting the WL of the image may adjust a brightness of the image, where a greater WL may increase the brightness of the image, and a smaller WL may decrease the brightness of the image.

For example, a first, narrower WW setting and a first, lower WL setting may be selected for visualizing a first segmented region (e.g., a brain), due to a relative softness and similarity of brain tissues. A second, wider WW setting and a second, higher WL setting may be selected for visualizing a second segmented region (e.g., a heart), due to a higher variance in tissues of the heart that include some harder tissues. A third narrower WW setting and a third, highest WL setting may be selected for visualizing a third segmented region (e.g., a pelvis), due to a relative hardness and similarity of bone tissues; and so on. Each of the first, second, and third WW and WL settings may be determined automatically, for example, using a histogram of intensity values within a segmented region and determining WW and WL based on some measure of the width and height of the histogram.

Optimizing the contrast at each of the segmented regions may also include adjusting kiloelectron voltage (keV) settings with which the image is displayed for each of the segmented regions. From an image data file generated during image reconstruction, two or more keV reference images may be generated with different keV settings (e.g., different sets of keV values). During post-reconstruction image processing, a visualization of the image with a desired set of keV values may be generated as a linear combination of the two or more keV reference images.

For example, a first segmented region may include a liver of the patient. An amount of contrast uptake by the liver may be lower than expected. The contrast uptake may be lower than expected due to a timing of the CT scan with respect to an injection of contrast agent into the patient's body, due to an insufficient amount of contrast being injected, and/or due to physiological factors of the patient (e.g., heart rate, respiratory rate, blood pressure, etc.). As a result of the contrast uptake by the liver being lower than expected, the image contrast within the liver in a reconstructed image may be lower than expected and/or desired. In response to the contrast being lower than desired, a keV image may be generated based on the linear combination of basis pair images for the first visualization, which may result in the liver being visualized with lower keV values than the reconstructed image. As a result of the lower keV values, a desired or ideal contrast of the first visualization of the liver may be higher than in the reconstructed image. The keV value for visualization with the desired contrast could be selected based on values within the segmented region and the Hounsfield unit curves across different keVs. (40, 45).

A second segmented region may include a kidney of the patient. An amount of contrast uptake by the kidney may be higher than expected. As a result of the contrast uptake by the kidney being higher than expected, a second opacity value of the kidney in the reconstructed image may be higher than expected and/or desired. In response to the second opacity value being higher than desired, a second visualization of the kidney may be generated based on a second linear combination of the two or more keV reference images, which may result in the kidney being visualized with higher keV values than the reconstructed image. As a result of the higher keV values, a desired or ideal contrast of the second visualization of the kidney may be higher than in the reconstructed image. For example, the second visualization may be based on keV values of (50, 60). In this way, by selecting different linear combinations of the two or more keV basis images, a set of keV values may be selected to visualize an anatomy of the patient in a desired (e.g., ideal) contrast.

In some embodiments, the linear combinations basis images can be used to generate different keV images for the first segmented region and the second segmented region. These keV images may be automatically generated based on a lookup table, with the keV values chosen based on the opacity values of the respective segmented regions.

Optimizing the contrast at each of the segmented regions may also include selecting different convolution algorithms (e.g., reconstruction kernels) to adjust frequency contents of projection data for each of the segmented regions, as part of a filtered back projection process during image reconstruction. Typically, a suitable kernel is selected based on a trade-off between a desired spatial resolution and a desired amount of noise, which may vary depending on a type of tissue being scanned. A kernel suitable for soft tissues (e.g., organs) may not be suitable for harder tissues (e.g., bone, cartilage, etc.), and vice versa. By using different kernels for different segmented regions, a contrast between anatomical features in each segmented region may be maximized.

Photon counting energy bins may be used for processing different anatomies included in the different segmented regions. For example, an energy bin could be chosen for a specific anatomy to optimize contrast level in that anatomy.

Optimizing the contrast at each of the segmented regions may include digitally adjusting the contrast in the image. In many cases, the contrast level of a segmented region may be digitally adjusted as a function of image data acquired at each voxel of a segmented region. In some embodiments, different algorithms or functions may be used to adjust the contrast level of different segmented regions. For example, a contrast level of a liver of the patient may be adjusted as a function of image data acquired at voxels of the image associated with the liver: a contrast level of a bone of the patient may be adjusted as a function of image data acquired at voxels of the image associated with the bone: a contrast level of a lung of the patient may be adjusted as a function of image data acquired at voxels of the image associated with the lung; and so on. In other words, a desired contrast for bone tissues may be different from a desired amount of contrast for liver tissues, which may be different from a desired amount of contrast for lung tissues.

At 408, method 400 includes generating a contrast-optimized image based on the contrast optimizations performed on each of the segmented regions, and method 400 ends.

Referring to FIG. 6, an image 600 is shown, where image 600 may be an MVI reconstructed from a scan without optimizing a contrast of different anatomies of image 600. The anatomies of image 600 have been visualized using a common set of visualization parameters post-reconstruction. Image 600 includes various anatomies of a patient, such as a lung 602, a liver 604, a kidney cortex 606, a spinal column 608, and a pelvis 610. In first image 600, the various anatomies are shown with different levels of contrast. Lung 602 is too dark to see any anatomical features within lung 602. Kidney cortex 606, spinal column 608, and pelvis 610 appear brighter than desirable. The brightness of kidney cortex 606 is indistinguishable from the brightness of spinal column 608 and pelvis 610, although the brightness of kidney cortex 606 is due to a high amount of contrast uptake, while the brightness of spinal column 608 and pelvis 610 is due to calcium in bone tissues. A contrast of liver 604 is too light to clearly see areas where the contrast agent has accumulated, such as an area 620, which may indicate a lesion. A darker liver 604 may be preferable, which may increase the contrast between liver 604 and area 620.

In contrast. FIG. 7 shows a contrast-optimized image 700 generated from image 600. Second image 700 includes lung 602, liver 604, kidney cortex 606, spinal column 608, and pelvis 610 of image 600. In contrast-optimized image 700, different visualization parameters have been applied post-reconstruction to adjust a contrast of the anatomies shown in FIG. 6. The different visualization parameters may include different WW and WL settings, different keV settings, different kernel energy levels, and/or other visualization parameters. Additionally, the contrast of image 700 may be digitally adjusted.

In contrast-optimized image 700, lung 602 has been visualized with a first set of visualization parameter settings, which may be different from the visualization parameter settings used in image 600. The first set of visualization parameter settings have increased a contrast of lung 602, such that anatomical features of lung 602 are now visible against lung tissues. Liver 604 has been visualized with a second set of visualization parameter settings, which may be different from the first set of visualization parameter settings and/or the visualization parameter settings used in image 600. The second set of visualization parameter settings have increased a contrast of liver 604, such that areas with higher than expected contrast uptake, such as area 620, are shown in greater contrast with liver tissues of liver 604 than in image 600. Kidney cortex 606 has been visualized with a third set of visualization parameter settings, which may be different from either or both of the first and second sets of visualization parameter settings and/or the visualization parameter settings used in image 600. As a result of using the third set of visualization parameters, the brightness of kidney cortex 606 has been reduced, making it easier to distinguish from other tissues. Spinal column 608 has been visualized with a fourth set of visualization parameter settings, which may be different from the first, second, and third sets of visualization parameter settings and/or the visualization parameter settings used in image 600. As a result of using the fourth set of visualization parameters, the brightness of spinal column 608 seen in image 600 has been reduced. Pelvis 610 has been visualized with a fifth set of visualization parameter settings, which may be different from the first, second, third, and fourth sets of visualization parameter settings and/or the visualization parameter settings used in image 600. As a result of using the fifth set of visualization parameters, the brightness of spinal column 608 seen in image 600 has been reduced. In this way, the visualization parameter settings may be customized for different segmented anatomies to generate a contrast-optimized image of higher image quality than an original reconstructed image that features a desired contrast at each of the different segmented anatomies.

As described below in reference to FIGS. 8-11, one or more colorized overlays may also be superimposed on contrast-optimized image 700 to further optimize a contrast of various anatomical regions of the patient and/or highlight certain aspects, features, or areas of the anatomical regions (e.g., using color).

Referring now to FIG. 8, a two-dimensional (2-D) contrast-optimized MVI 800 is shown, where contrast-optimized MVI 800 may be reconstructed from projection data of a patient acquired via a CT imaging system, such as imaging systems 100 and 200 of FIGS. 1 and 2A. For example, contrast-optimized MVI 800 may be a contrast-optimized 120 keV image. Contrast-optimized MVI 800 and the other images and visualizations described herein may be displayed on a display device of the CT imaging system or a visualization system of the CT imaging system (e.g., operator console 234 or display device 272, respectively), to be read by a radiologist, for example, to determine a presence or extent of diseased tissues of the patient.

Contrast-optimized MVI 800 shows a relative contrast of tissues of various anatomical structures of the patient, as measured in Hounsfield units at each voxel/pixel of contrast-optimized MVI 800. The relative contrast between the tissues of the various anatomical structures shown in contrast-optimized MVI 800 is a result of differing degrees of contrast agent uptake at the various anatomical structures. Additionally, pixel intensity values of contrast-optimized MVI 800 may have been adjusted based on a set of visualization parameters, to maximize the relative contrast between the tissues of the various anatomical structures throughout the entire image, as described above.

Darker portions of contrast-optimized MVI 800 may correspond to areas of low contrast uptake. Areas of high contrast uptake may be shown as brighter spots or portions of contrast-optimized MVI 800, which may indicate a presence of high metabolic activity often associated with diseased tissues. For example, a plurality of bright spots 808 may be seen on a liver 806 of the patient, which may be diseased tissues. Similarly, a bright portion 812 of a kidney 810 may be indicative of diseased tissues. During the contrast-optimization process (e.g., method 300), relative contrasts between bright spots 808 and liver 806 and between bright portion 812 and kidney 810 may have been adjusted using different display parameters for liver 806 and kidney 810.

However, healthy harder tissues such as bone tissues may also appear as bright spots, such as portions 802 and 804 of a spine of the patient. Thus, a drawback of contrast-optimized MVI 800 is that harder, bone tissues of the patient may distract a viewer and/or make it harder to identify areas of diseased tissue.

FIG. 9 shows a colorized heat map 900 generated from the same image data used to reconstruct contrast-optimized MVI 800 of FIG. 8. Heap map 900 may be generated by material decomposition using two or more basis materials. Specifically, heat map 900 shows areas of high iodine absorption in red, where iodine is the contrast agent injected into the patient prior to performing the scan. The areas of high iodine absorption include the spots 808 on liver 806 and portion 812 of kidney 810, which may be diseased tissues. A contrast between the potentially diseased tissues at spots 808 and portion 812 and healthy tissues of liver 806 and kidney 810, respectively, may be greater than in contrast-optimized MVI 800, whereby the diseased tissues may be more easily distinguished from the healthy tissues in heat map 900 than in contrast-optimized MVI 800.

However, in heat map 900, the similarity in appearance between the harder bone tissues and diseased soft tissues is also apparent, where portions 802 and 804 of the spine are shown in red. As with contrast-optimized MVI 800, the visual similarity between the bone tissues and the diseased tissues may make it harder for a radiologist to read heat map 900.

FIG. 10 shows a first visualization 1000 of contrast-optimized MVI 800, where first visualization 1000 includes a first color overlay superimposed on contrast-optimized MVI 800 of FIG. 8. The first color overlay may be generated from the same projection data used to reconstruct contrast-optimized MVI 800 of FIG. 8 and heat map 900 of FIG. 9. The first color overlay may be generated by material decomposition, in accordance with the procedure described above in reference to method 300 of FIG. 3. In some embodiments, the first color overlay may also be contrast-optimized via a similar procedure to the procedure used to optimize the contrast of MVI 800. For example, the first color overlay may be contrast-optimized by following one or more steps of method 400 of FIG. 4.

The first color overlay applies color to segmented regions, similar to heat map 900, to highlight a contrast between healthy soft tissues and diseased soft tissues of an abdomen of the patient. In FIG. 10, healthy tissues of liver 806 and kidney are shown in green, while the potentially diseased tissues corresponding to spots 808 and portion 812 are shown in yellow. The contrast between the healthy tissues and the potentially diseased tissues may be greater in first visualization 1000 than in either of contrast-optimized MVI 800 and heat map 900. Thus, by including the first color overlay, the potentially diseased tissues may be more easily discerned and distinguished from healthy tissues in first visualization 1000 than in contrast-optimized MVI 800.

In various embodiments, the diseased soft tissues may be identified, distinguished, and/or segmented using a tissue assessment DL model trained to assess tissues and classify the tissues as diseased or healthy. In one embodiment, the DL model is a neural network model trained on reconstructed images including diseased and/or healthy tissues, using ground truth images where diseased tissues have been segmented manually or by a different procedure. For example, the neural network model may be a convolutional neural network (CNN) including a plurality of different layers, such as the 3D U-net. In other embodiments, the neural network may be a generative adversarial network (GAN), or a different type of neural network.

Additionally, in contrast to heat map 900, the first color overlay colorizes the areas of high iodine concentration in the soft tissues of the abdomen, but does not colorize the harder, bone tissues of the patient's spine. In other words, portions 802 and 804 of the spine of the patient shown in red in heat map 900 are not colored in the first color overlay. Because the first color overlay is superimposed on contrast-optimized MVI 800 in first visualization 1000, areas of the spine are unchanged from contrast-optimized MVI 800. As a result of the first color overlay not colorizing the spine, the contrast between the healthy soft tissues of liver 806 and kidney 810 and the diseased tissues at spot 808 and 812, respectively, may be more salient in visualization 1000 than in contrast-optimized MVI 800 and heat map 900, making first visualization 1000 easier to read. In this way, a visual saliency of various anatomical regions may be adjusted to facilitate drawing the attention of a reader (e.g., a radiologist) to areas of interest.

FIG. 11 shows a second visualization 1100 of contrast-optimized MVI 800, where second visualization 1100 includes the first color overlay of first visualization 1000 superimposed on contrast-optimized MVI 800 of FIG. 8. Additionally, second visualization 1100 includes a second color overlay generated from the same image data used to reconstruct contrast-optimized MVI 800 of FIG. 8 and heat map 900 of FIG. 9. The second color overlay may also be generated by material decomposition, in accordance with the procedure described above in reference to method 300 of FIG. 3.

While the first color overlay of first visualization 1000 applies color to the segmented regions to highlight the contrast between healthy soft tissues and diseased soft tissues of the abdomen of the patient, the second color overlay shown in second visualization 1100 colorizes bone tissues of the spine not colored by the first color overlay. Specifically, the second color overlay includes colorized data showing relative water densities within the bone. In FIG. 11, colorized portions 802 and 804 shows lower relative water densities in different hues of orange, where higher water densities are shown in green.

In various embodiments, the more dense (e.g., harder) bone cortex tissues and the less dense (e.g., softer) bone marrow tissues may be identified, distinguished, and/or segmented using a bone marrow segmentation model trained to segment different types of bone tissues. In one embodiment, the bone marrow segmentation model is a DL neural network model trained on reconstructed images including harder and softer bone tissues, using ground truth images where bone cortex and/or bone marrow tissues have been segmented manually or by a different procedure. The DL neural network model may be based on 3D U-net architecture. In other embodiments the model may be a CNN, a GNN, or a different type of neural network.

The second color overlay additionally shows information about healthy and diseased bone tissues. In some embodiments, the information may be obtained using a trained tissue assessment DL model, as described above. In other embodiments, such as the embodiment depicted in FIG. 11, the information may be obtained from water-hydroxyapatite (HAP) images. The water-HAP image distinguishes between areas of bone marrow with lower water concentration in the spine (802 and 804), which are shown in orange in FIG. 11, and areas of higher water concentration, such as areas 1102 and 1104, which are shown in green. Areas 1102 and 1104 may indicate bone marrow edema, where fluid has accumulated as a result of inflammation, trauma, or a different reason. In some embodiments, a color assigned to each pixel may be based on a WW/WL used for the visualization.

As a result of superimposing both of the first color overlay and the second color overlay on contrast-optimized MVI 800, the second visualization may allow a radiologist reading contrast-optimized MVI 800 to more easily distinguish both the contrast between the healthy soft tissues of liver 806 and kidney 810 and the diseased tissues at spot 808 and 812, and the contrast between the areas of high fluid concentration 1102 and 1104 from healthy bone tissues of the spine, such as in portions 802 and 804. Thus, the radiologist may efficiently read multiple anatomies of a patient using second visualization 1100, and may not have to review additional images.

In an alternative scenario without second visualization 1100, the radiologist might first read contrast-optimized MVI 800, to determine a presence and/or an extent of diseased soft tissues. However, the areas of high fluid concentration 1102 and 1104 are not visible in contrast-optimized MVI 800. The radiologist may then view a MD image such as heat map 900, which may show an increased contrast between the diseased soft tissues and healthy soft tissues. The radiologist may then view the water-HAP image, which may show the areas of high fluid concentration 1102 and 1104, but may not adequately distinguish between the diseased soft tissues and healthy soft tissues. Thus, in the alternative scenario where second visualization 1100 is not available, the radiologist may rely on three different images to diagnose the patient, rather than one. As a result of using second visualization 1100, a workflow of the radiologist may be simplified, and an amount of time spent by the radiologist reviewing images of the patient may be decreased.

Further, a first amount of memory and/or processing resources of the CT system consumed during the generation of second visualization 1100 may be less than a second amount of memory and/or processing resources of the CT system consumed during the generation of an MVI, a MD image, and a water-HAP image. For example, during the generation of each of the MVI. MD image, and water-HAP image, the radiologist may experiment with various display parameter settings to attempt to optimize a contrast between diseased tissues and healthy tissues. Experimenting with the various display parameters may increase an overall amount of processing performed using the CT system, and an amount of time spent by the radiologist using the CT system, during which less memory and/or processing resources of the CT system are available for performing other tasks. Alternatively, second visualization 1100 may be generated more quickly and efficiently, relying on tested mathematical formulas for maximizing relative contrasts between different anatomical features rather than a cumbersome trial-and-error process. Additionally, in the alternative scenario, even if the MVI. MD image, and water-HAP image generated manually by the radiologist were combined into a single image in a different manner, a first relative contrast between the different anatomical features may be less desirable than a second relative contrast between the different anatomical features in second visualization 1100. Because second visualization 1100 can be generated more rapidly and efficiently, using less processing and/or memory resources of the CT system, an overall functioning of the CT system may be increased as a result of the systems and methods described herein.

Turning now to FIG. 12, an exemplary view of an automated report 1200 is shown, where automated report 1200 may be generated by an image visualization system such as image visualization system 250 of FIG. 2B. Specifically, automated report 1200 may be generated by a visualization module of the image visualization system, such as visualization module 264. Automated report 1200 may show and/or describe a comparison of two or more visualizations of one or more anatomical regions of a patient on which a CT scan has been performed, along with other textual or visual information. In particular, the two or more visualizations may include a first visualization generated at a first time, and a second visualization generated at a later time. Thus, a comparison of the first visualization with the second visualization may show a trend in the CT image data, such as a progression of a disease. Automated report 1200 may be generated as a result of a process such as method 300 described above in reference to FIG. 3.

Automated report 1200 includes a patient identification panel 1202, which may include information such as name of the patient, date of birth of the patient, the date of the automated report, and/or relevant descriptive information about the report, such as a clinical history of the patient. Automated report 1200 includes a contrast-optimized image 1204, which may be generated in accordance with method 300 described above. In FIG. 12, contrast-optimized image 1204 includes a portion of a bone of the patient. In other embodiments, contrast optimized image 1204 may include a different anatomical region of the patient.

Automated report 1200 also includes an enhanced visualization 1206 of contrast-optimized image 1204, where enhanced visualization 1206 shows a first color overlay superimposed on contrast-optimized image 1204. The first color overlay colorizes portions of the bone using a color gradient between blue and green, where blue may represent healthy tissues, and green may represent diseased tissues. In various embodiments, contrast-optimized image 1204 is reconstructed from scan data of the patient, and the first color overlay is generated from a MD image reconstructed from the same scan data. By superimposing the first color overlay on contrast-optimized image 1204, the diseased tissues highlighted in green are easier to distinguish from the healthy tissues highlighted in blue than in contrast-optimized image 1204.

Automated report 1200 further includes a previous visualization 1208 of the bone of the patient, where previous visualization 1208 was generated from scan data acquired at an earlier CT scan performed on Mar. 23, 2019. Previous visualization 1208 includes a second color overlay similar to the first color overlay, where the second color overlay shows diseased tissues in green and healthy tissues in blue. Previous visualization 1208 is displayed side-by-side with an enlarged view 1210 of enhanced visualization 1206 (e.g., where previous visualization 1208 and enlarged view 1210 are the same size), which corresponds to an exam date of Jul. 7, 2019. When displayed side-by-side, it can be seen that a first area of the diseased tissues shown in green in enlarged view 1210 (e.g., enhanced visualization 1206) has a smaller area (e.g., volume) than a second area of the diseased tissues shown in previous visualization 1208. A first volume of the diseased tissues in enlarged view 1210 is indicated by a label 1222, and a second volume of the diseased tissues in previous visualization 1208 is indicated by a label 1220. In accordance with labels 1220 and 1222, automated report 1200 shows that the volume of the lesion has decreased from 13.6 mm³ to 1.8 mm³, suggesting that the patient is improving. Additionally, automated report 1200 includes a bar graph 1216, which shows a difference in the bone edema volume between the exam dated March 23 and the exam dated July 7.

Based on a calculation of the difference in volume between the diseased tissues of enhanced visualization 1206/enlarged view 1210 and the diseased tissues of previous visualization 1208, a progression of the disease indicated by the diseased tissues can be estimated, which is described in a findings panel 1214. Findings panel 1214 may further include additional information determined and/or calculated by the image visualization system, such as precise measurements, descriptive text, areas of concern, etc. In some embodiments, the progression of the disease and the additional information may be summarized in natural language in findings panel 1214, where the natural language is generated by one or more AI models, such as a rules-based model and/or a neural network model.

By providing automated report 1200 to a radiologist, the radiologist may more easily assess an extent of the diseased tissues in the patient and differentiate between the diseased tissues and the healthy tissues in contrast-optimized image 1204 and enhanced visualization 1206 than in a non-contrast-optimized image typically reconstructed by the CT system. In an alternate scenario in which contrast-optimized image 1204 is not available, the radiologist would find it more difficult to distinguish between the healthy and diseased tissues in the non-contrast-optimized image, due to a lack of contrast between the healthy tissues and the diseased tissues. Further, the radiologist would likely first view the non-contrast-optimized image, and upon finding it more difficult to distinguish between the healthy and diseased tissues, the radiologist may subsequently reconstruct and view one or more MD images to determine the extent of the diseased tissues. As a result of having to view multiple images, a workflow of the radiologist would be more cumbersome and time-consuming in the alternate scenario in which contrast-optimized image 1204 is not available.

Additionally, by displaying the enhanced visualization 1206 side-by-side with previous visualization 1208, the radiologist may quickly and efficiently determine a change in the volume of the diseased tissues since the last exam date. In the alternate scenario where contrast-optimized image 1204 and enhanced visualization 1206 are not available, the radiologist would have to search for a previous reconstructed image from the previous exam in a PACS system of the CT system, and alternate between displaying the previous reconstructed image on the display screen and displaying a current reconstructed image from a current exam on the display screen. As a result of not being able to view the previous reconstructed image and the current reconstructed image side-by-side, it may take longer to find an appropriate previous reconstructed image to compare with the current reconstructed image. Additionally, it may be difficult to compare the extent of the diseased tissues between the previous reconstructed image in the current reconstructed, whereby a first assessment of the progression of the disease may be less accurate and more time-consuming than a second assessment of the progression of the disease using automated report 1200.

The technical effect of generating a visualization including a contrast-optimized image with colorized overlays showing different anatomical regions and tissues with different properties is that a radiologist may more quickly and efficiently diagnose a patient by viewing the visualization than by viewing multiple reconstructed images of the patient showing different aspects of the anatomical regions, reducing a usage of a CT system and its computational resources. The technical effect of automatically generating a report including a first visualization generated from a current scan and a second visualization of the same patient from an earlier scan is that a progression of a disease of the patient may be assessed by comparing the first visualization with the second visualization in a side-by-side manner.

The disclosure also provides support for a method for a computed tomography (CT) system, the method comprising: performing a CT scan of a patient injected with a contrast agent, reconstructing an image based on projection data acquired during the CT scan, adjusting a first contrast of a first anatomical region of the image based on a first set of display parameter settings of the CT system, adjusting a second contrast of a second anatomical region of the image based on a second set of display parameter settings of the CT system, the second anatomical region different from the first anatomical region, the second set of display parameter settings different from the first set of display parameter settings, displaying a contrast-optimized image on a display device of the CT system, the contrast-optimized image showing the first anatomical region of the image in the first contrast, and the second anatomical region of the image in the second contrast, the second contrast different from the first contrast. In a first example of the method, the method further comprises: performing an automated segmentation of a plurality of anatomical regions of the reconstructed image, assessing a level of contrast agent uptake at a respective plurality of anatomical reference points of the plurality of segmented anatomical regions, adjusting the first set of display parameter settings based on a first level of contrast agent uptake at a first anatomical reference point of the first anatomical region, and adjusting the second set of display parameter settings based on a second level of contrast agent uptake at a second anatomical reference point of the second anatomical region. In a second example of the method, optionally including the first example, the first set of display parameter settings includes a first window width (WW) setting and a first window level (WL) setting, and the second set of display parameter settings includes a second WW setting and a second WL setting, where the second WW setting is different from the first WW setting and the second WL setting is different from the first WL setting. In a third example of the method, optionally including one or both of the first and second examples: the first set of display parameter settings includes a first kiloelectron voltage (keV) setting based on a first linear combination of a first basis image reconstructed with a first set of keV values and a second basis image reconstructed with a second set of keV values, and the second set of display parameter settings includes a second keV setting based on a second linear combination of the first basis image and the second basis image. In a fourth example of the method, optionally including one or more or each of the first through third examples, the method further comprises: calculating opacity values of the segmented first and second anatomical regions, and retrieving the first keV setting and the second keV setting from a lookup table stored in a memory of the CT system based on the respective calculated opacity values. In a fifth example of the method, optionally including one or more or each of the first through fourth examples: adjusting the first set of display parameter settings further comprises adjusting frequency contents of a first set of projection data of the first anatomical region based on a first kernel, and adjusting the second set of display parameter settings further comprises adjusting frequency contents of a second set of projection data of the second anatomical region based on a second kernel, the second kernel different from the first kernel. In a sixth example of the method, optionally including one or more or each of the first through fifth examples: adjusting the first set of display parameter settings further comprises adjusting the first contrast of the first anatomical region based on a first photon count at a first photon counting energy bin associated with the first anatomical region, and adjusting the second set of display parameter settings further comprises adjusting the second contrast of the second anatomical region based on a second photon count at a second photon counting energy bin associated with the second anatomical region. In a seventh example of the method, optionally including one or more or each of the first through sixth examples, adjusting the first set of display parameter settings further comprises digitally adjusting the first contrast of the first anatomical region as a function of a first set of image data acquired at each voxel of the first anatomical region, and adjusting the second set of display parameter settings further comprises adjusting the second contrast of the second anatomical region as a function of a second set of image data acquired at each voxel of the second anatomical region. In a eighth example of the method, optionally including one or more or each of the first through seventh examples, the method further comprises: displaying a visualization of material decomposition information acquired from the patient during the CT scan superimposed on the contrast-optimized image, the visualization of the material decomposition information including one or more colorized overlays generated based on the material decomposition information, the one or more colorized overlays including anatomical regions having a 1:1 correspondence with anatomical regions of the contrast-optimized image with respect to size and positioning, the one or more colorized overlays applying different colors to different anatomical regions of the reconstructed image. In a ninth example of the method, optionally including one or more or each of the first through eighth examples, a first colorized overlay of the one or more colorized overlays shows healthy tissues of the patient in a first color, and shows diseased tissues of the patient in a second color, wherein the first color and the second color highlight a contrast between the healthy tissues and the diseased tissues of the patient. In a tenth example of the method, optionally including one or more or each of the first through ninth examples, the method further comprises: generating an automated report comparing the contrast-optimized image including the one or more colorized overlays with a second contrast-optimized image including a second set of colorized overlays, the second contrast-optimized image generated from previous scan data stored in a picture archiving and communications system (PACS) coupled to the CT system, the automated report describing at least a progression of a disease in an anatomical region of the patient, the progression of the disease determined by comparing a first size of a first area of diseased tissue in the contrast-optimized image with a second size of a second area of diseased tissue in the second contrast-optimized image. In a eleventh example of the method, optionally including one or more or each of the first through tenth examples, describing the progression of the disease in the anatomical region of the patient further comprises displaying the contrast-optimized and the second contrast-optimized image side by side in the automated report. In a twelfth example of the method, optionally including one or more or each of the first through eleventh examples, the automated report includes a summary of the progression of the disease and additional information in natural language.

The disclosure also provides support for a computed tomography (CT) system, comprising a processor and a non-transitory memory including instructions that when executed, cause the processor to: reconstruct an image based on scan data of a patient acquired during a CT scan of the CT system, segment a plurality of anatomical regions of the reconstructed image, assess an organ perfusion status of a contrast agent injected into the patient prior to the CT scan at a respective plurality of anatomical reference points of the plurality of anatomical regions, based on the assessed organ perfusion status of the contrast agent, determine an ideal contrast of each of the segmented anatomical regions, adjust display parameter settings of the CT system individually for each of the segmented anatomical regions, to generate a contrast-optimized image showing each of the segmented anatomical regions in a corresponding ideal contrast, and display the contrast-optimized image on a display device of the CT system. In a first example of the system, a first ideal contrast of a first anatomical region of the plurality of anatomical regions is different from a second ideal contrast of a second anatomical region of the plurality of anatomical regions. In a second example of the system, optionally including the first example, the display parameter settings include: a window width setting and/or a window level setting for displaying a respective segmented anatomical region, a keV setting for displaying the respective segmented anatomical region, a kernel to apply to adjust frequency contents of projection data of the respective segmented anatomical region, a contrast of the respective segmented anatomical region as a function of material decomposition information associated with the respective segmented anatomical region, and a contrast of the respective segmented anatomical region as a function of image data of each voxel of the respective segmented anatomical region. In a third example of the system, optionally including one or both of the first and second examples, further instructions are stored in the non-transitory memory that when executed, cause the processor to display a colorized overlay superimposed on the contrast-optimized image, the colorized overlay showing healthy tissues of the patient in a first color, and diseased tissues of the patient in a second color, the colorized overlay based on material decomposition data generated from the CT scan. In a fourth example of the system, optionally including one or more or each of the first through third examples, further instructions are stored in the non-transitory memory that when executed, cause the processor to generate an automated report comparing the contrast-optimized image with a second contrast-optimized image, the second contrast-optimized image generated from previous scan data stored in a picture archiving and communications system (PACS) coupled to the CT system, the automated report showing a progression of a disease in an anatomical region of the patient, the progression of the disease determined by comparing a first size of a first area of diseased tissue in the contrast-optimized image with a second size of a second area of diseased tissue in the second contrast-optimized image.

The disclosure also provides support for a method for visualizing a progression of a disease of a patient, the method comprising: performing a scan of the patient using a computed tomography (CT) system, and reconstructing a first image from projection data acquired during the scan, adjusting display parameter settings of the CT system individually for each of a plurality of anatomical regions of the patient, to generate a first contrast-optimized image showing each anatomical region of the plurality of anatomical regions in an ideal contrast, the ideal contrast based on an assessed organ perfusion status of a contrast agent at the anatomical region, retrieving a second image of the patient from a picture archiving and communications system (PaCS) coupled to the CT system, adjusting the display parameter settings of the CT system individually for each of the plurality of anatomical regions of the patient in the second image, to generate a second contrast-optimized image showing each anatomical region of the plurality of anatomical regions in the ideal contrast, comparing a first size of a first area of diseased tissue in the first contrast-optimized image with a second size of a second area of diseased tissue in the second contrast-optimized image to determine the progression of the disease, generating a visualization showing the first contrast-optimized image side-by-side with the second contrast-optimized image, the visualization including a measured difference between the first size and the second size, sending an automated report including the visualization to a user of the CT system. In a first example of the method, the first contrast-optimized image and the second contrast-optimized image include one or more superimposed colorized overlays showing healthy tissues of the patient in a first color, and diseased tissues of the patient in a second color, the one or more superimposed colorized overlays based on material decomposition data generated from the scan.

The disclosure also provides support for a method for a computed tomography (CT) system, the method comprising: performing a CT scan of a patient injected with a contrast agent, reconstructing a monochromatic virtual image (MVI) based on projection data acquired during the CT scan, generating a first contrast-optimized image based on the MVI, the first contrast-optimized image showing a plurality of anatomical regions, each anatomical region displayed using a different set of display parameters selected to maximize a contrast between different anatomical features of the anatomical region, reconstructing a first basis material decomposition (MD) image based on the acquired projection data, the first MD image including anatomical regions having a 1:1 correspondence to anatomical regions of the first contrast-optimized image with respect to size and positioning, generating one or more colorized overlays from the first MD image, each colorized overlay applying one or more colors to the anatomical region to show spectral decomposition information relating to the anatomical region, superimposing the one or more colorized overlays on the first contrast-optimized image, and displaying the first contrast-optimized image including the one or more colorized overlays on a display screen of the CT system. In a first example of the method, generating the first contrast-optimized image based on the MVI further comprises: segmenting the plurality of anatomical regions of the MVI, assessing an organ perfusion status of the contrast agent at a respective plurality of anatomical reference points of the plurality of segmented anatomical regions, based on the assessed organ perfusion status of the contrast agent, determining an ideal contrast of each of the segmented anatomical regions, adjusting display parameter settings of the CT system individually for each of the segmented anatomical regions. In a second example of the method, optionally including the first example: a first colorized overlay of the one or more colorized overlays applies a first set of colors to a first anatomical region, and applies no color to a second anatomical region, and a second colorized overlay of the one or more colorized overlays applies no color to the first anatomical region, and applies a second set of colors to the second anatomical region. In a third example of the method, optionally including one or both of the first and second examples, the second set of colors is different from the first set of colors. In a fourth example of the method, optionally including one or more or each of the first through third examples, the method further comprises: using a tissue assessment deep learning (DL) model to identify diseased tissues in one or both of the first contrast-optimized image and the first MD image, wherein the first colorized overlay shows healthy tissues of the first anatomical region in a first color, and shows the identified diseased tissues of the first anatomical region in a second color, wherein the first color and the second color are selected to highlight a contrast between the healthy tissues and the diseased tissues. In a fifth example of the method, optionally including one or more or each of the first through fourth examples, the tissue assessment DL model is a neural network trained on healthy and diseased tissue types of the first anatomical region. In a sixth example of the method, optionally including one or more or each of the first through fifth examples, the first anatomical region includes bone tissues. In a seventh example of the method, optionally including one or more or each of the first through sixth examples, the method further comprises: using a bone marrow segmentation model to segment the bone tissues into portions of different densities, wherein the first colorized overlay uses color to distinguish between the different segmented portions. In a eighth example of the method, optionally including one or more or each of the first through seventh examples, at least one of the tissue assessment DL model and the bone marrow segmentation model take image data from a water-hydroxyapatite (HAP) image as input. In a ninth example of the method, optionally including one or more or each of the first through eighth examples, the bone marrow segmentation model includes a neural network trained on bone tissue in spectral CT images. In a tenth example of the method, optionally including one or more or each of the first through ninth examples, the method further comprises: retrieving a second MVI of the patient generated from a previous scan and stored in a picture archiving and communications system (PACS) coupled to the CT system, generating a second contrast-optimized image based on the second MVI, using a same set of display parameters selected for generating the first contrast-optimized image, reconstructing a second MD image based on projection data used to generate the second MVI, generating a second set of colorized overlays from the second MD image, using a same procedure used to generate the one or more colorized overlays from the first MD image, generating an automated report including both of the first contrast-optimized image and the second contrast-optimized image, the automated report describing at least a progression of a disease in an anatomical region of the patient, the progression of the disease determined by comparing a first size of a first area of diseased tissue in the first contrast-optimized image with a second size of a second area of diseased tissue in the second contrast-optimized image, and sending the report to a user of the CT system. In an eleventh example of the method, optionally including one or more or each of the first through tenth examples, the description of the progression of the disease in the automated report includes a textual description of a measured difference between the first area of diseased tissue and the second area of diseased tissue. In a twelfth example of the method, optionally including one or more or each of the first through eleventh examples, the description of the progression of the disease automated report includes a graphic depicting the first contrast-optimized image and the second contrast-optimized image side by side.

The disclosure also provides support for a computed tomography (CT) system, comprising a processor and a non-transitory memory including instructions that when executed, cause the processor to: reconstruct a monochromatic virtual image (MVI) and a basis material decomposition (MD) image based on scan data acquired during a CT scan performed on a patient using the CT system, segment a plurality of anatomical regions of the MVI and the MD image, adjust display parameter settings of the CT system individually for each of the segmented anatomical regions of the MVI, to generate a contrast-optimized MVI showing each of the segmented anatomical regions in a corresponding ideal contrast, generate a visualization of the contrast-optimized MVI including one or more color overlays superimposed on the contrast-optimized MVI, the color overlays generated from the MD image, the color overlays applying color to portions of the segmented anatomical regions, compare the visualization with a previous visualization of the patient generated from a previous scan to determine a progression of a disease of the patient, generate an automated report describing the progression, the automated report including the visualization and the previous visualization, and send the automated report to a user of the CT system and/or display the visualization on a display device of the CT system. In a first example of the system, the one or more color overlays show healthy tissues in a first color, and diseased tissues in a second color, the diseased tissues distinguished from the healthy tissues using a tissue assessment deep learning (DL) model. In a second example of the system, optionally including the first example, the one or more color overlays show harder bone tissues in a first color, and softer bone tissues in a second color, the harder bone tissues distinguished from the softer bone tissues based on relative water densities of the harder bone tissues and the softer bone tissues. In a third example of the system, optionally including one or both of the first and second examples, further instructions are stored in the non-transitory memory that when executed, cause the processor to generate the previous visualization from a prior study of the patient stored in a picture archiving and communications system (PACS) coupled to the CT system, the previous visualization generated by following a same procedure used to generate the visualization from the MVI and the MD image.

The disclosure also provides support for a method for a computed tomography (CT) system, the method comprising: injecting a contrast agent into a patient, performing a CT scan on the patient, reconstructing a monochromatic virtual image (MVI) and a basis material decomposition (MD) image based on scan data acquired during the CT scan, segmenting a plurality of anatomical regions of the reconstructed MVI and the reconstructed MD image, performing an assessment of an absorption of the contrast agent at each segmented anatomical region of the plurality of anatomical regions, based on the reconstructed MVI, adjusting a contrast of each segmented anatomical region, by adjusting a set of display parameter settings of the CT system separately for each segmented anatomical region of the reconstructed MVI, generating a visualization of the reconstructed MVI showing the adjusted contrast of each segmented anatomical region, superimposing one or more colorized overlays on the visualization, the colorized overlays generated from the MD image, the colorized overlays applying color to portions of the segmented anatomical regions, comparing the visualization with a previous visualization of the patient generated from a previous scan to determine a progression of a disease of the patient, generating an automated report describing the progression, the automated report including the visualization and the previous visualization, and sending the automated report to a user of the CT system. In a first example of the method, adjusting the set of display parameter settings of the CT system separately for each segmented anatomical region of the reconstructed MVI further comprises: for each segmented anatomical region, at least one of: adjusting a window width setting and/or a window level setting of the CT system, adjusting a keV setting of the CT system for displaying the MVI, selecting a kernel to apply to adjust frequency contents of projection data of the segmented anatomical region, adjusting a contrast of the segmented anatomical region based on spectral information associated with the segmented anatomical region, digitally adjusting the contrast of the segmented anatomical region as a function of image data of each voxel of the segmented anatomical region. In a second example of the method, optionally including the first example, the automated report shows the visualization and the previous visualization side by side, and includes a textual description of a measured difference between a first area of diseased tissue of the visualization and a second area of diseased tissue of the previous visualization.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for a computed tomography (CT) system, the method comprising:
performing a CT scan of a patient injected with a contrast agent;
reconstructing an image based on projection data acquired during the CT scan;
adjusting a first contrast of a first anatomical region of the image based on a first set of display parameter settings of the CT system;
adjusting a second contrast of a second anatomical region of the image based on a second set of display parameter settings of the CT system, the second anatomical region different from the first anatomical region, the second set of display parameter settings different from the first set of display parameter settings;
displaying a contrast-optimized image on a display device of the CT system, the contrast-optimized image showing the first anatomical region of the image in the first contrast, and the second anatomical region of the image in the second contrast, the second contrast different from the first contrast.

2. The method of claim 1, further comprising:
performing an automated segmentation of a plurality of anatomical regions of the reconstructed image;
assessing a level of contrast agent uptake at a respective plurality of anatomical reference points of the plurality of segmented anatomical regions;
adjusting the first set of display parameter settings based on a first level of contrast agent uptake at a first anatomical reference point of the first anatomical region; and
adjusting the second set of display parameter settings based on a second level of contrast agent uptake at a second anatomical reference point of the second anatomical region.

3. The method of claim 2, wherein the first set of display parameter settings includes a first window width (WW) setting and a first window level (WL) setting, and the second set of display parameter settings includes a second WW setting and a second WL setting, where the second WW setting is different from the first WW setting and the second WL setting is different from the first WL setting.

4. The method of claim 2, wherein:
the first set of display parameter settings includes a first kiloelectron voltage (keV) setting based on a first linear combination of a first basis image reconstructed with a first set of keV values and a second basis image reconstructed with a second set of keV values; and
the second set of display parameter settings includes a second keV setting based on a second linear combination of the first basis image and the second basis image.

5. The method of claim 4, further comprising calculating opacity values of the segmented first and second anatomical regions, and retrieving the first keV setting and the second keV setting from a lookup table stored in a memory of the CT system based on the respective calculated opacity values.

6. The method of claim 2, wherein:
adjusting the first set of display parameter settings further comprises adjusting frequency contents of a first set of projection data of the first anatomical region based on a first kernel; and
adjusting the second set of display parameter settings further comprises adjusting frequency contents of a second set of projection data of the second anatomical region based on a second kernel, the second kernel different from the first kernel.

7. The method of claim 2, wherein:
adjusting the first set of display parameter settings further comprises adjusting the first contrast of the first anatomical region based on a first photon count at a first photon counting energy bin associated with the first anatomical region; and
adjusting the second set of display parameter settings further comprises adjusting the second contrast of the second anatomical region based on a second photon count at a second photon counting energy bin associated with the second anatomical region.

8. The method of claim 2, wherein:
adjusting the first set of display parameter settings further comprises digitally adjusting the first contrast of the first anatomical region as a function of a first set of image data acquired at each voxel of the first anatomical region; and
adjusting the second set of display parameter settings further comprises adjusting the second contrast of the second anatomical region as a function of a second set of image data acquired at each voxel of the second anatomical region.

9. The method of claim 1, further comprising displaying a visualization of material decomposition information acquired from the patient during the CT scan superimposed on the contrast-optimized image, the visualization of the material decomposition information including one or more colorized overlays generated based on the material decomposition information, the one or more colorized overlays including anatomical regions having a 1:1 correspondence with anatomical regions of the contrast-optimized image with respect to size and positioning, the one or more colorized overlays applying different colors to different anatomical regions of the reconstructed image.

10. The method of claim 9, wherein a first colorized overlay of the one or more colorized overlays shows healthy tissues of the patient in a first color, and shows diseased tissues of the patient in a second color, wherein the first color and the second color highlight a contrast between the healthy tissues and the diseased tissues of the patient.

11. The method of claim 10, further comprising:
generating an automated report comparing the contrast-optimized image including the one or more colorized overlays with a second contrast-optimized image including a second set of colorized overlays, the second contrast-optimized image generated from previous scan data stored in a picture archiving and communications system (PACS) coupled to the CT system, the automated report describing at least a progression of a disease in an anatomical region of the patient, the progression of the disease determined by comparing a first size of a first area of diseased tissue in the contrast-optimized image with a second size of a second area of diseased tissue in the second contrast-optimized image.

12. The method of claim 11, wherein describing the progression of the disease in the anatomical region of the patient further comprises displaying the contrast-optimized and the second contrast-optimized image side by side in the automated report.

13. The method of claim 11, wherein the automated report includes a summary of the progression of the disease and additional information in natural language.

14. A computed tomography (CT) system, comprising a processor and a non-transitory memory including instructions that when executed, cause the processor to:
reconstruct an image based on scan data of a patient acquired during a CT scan of the CT system;
segment a plurality of anatomical regions of the reconstructed image;
assess an organ perfusion status of a contrast agent injected into the patient prior to the CT scan at a respective plurality of anatomical reference points of the plurality of anatomical regions;
based on the assessed organ perfusion status of the contrast agent, determine an ideal contrast of each of the segmented anatomical regions;
adjust display parameter settings of the CT system individually for each of the segmented anatomical regions, to generate a contrast-optimized image showing each of the segmented anatomical regions in a corresponding ideal contrast; and
display the contrast-optimized image on a display device of the CT system.

15. The CT system of claim 14, wherein a first ideal contrast of a first anatomical region of the plurality of anatomical regions is different from a second ideal contrast of a second anatomical region of the plurality of anatomical regions.

16. The CT system of claim 14, wherein the display parameter settings include:
a window width setting and/or a window level setting for displaying a respective segmented anatomical region;
a keV setting for displaying the respective segmented anatomical region;
a kernel to apply to adjust frequency contents of projection data of the respective segmented anatomical region;
a contrast of the respective segmented anatomical region as a function of material decomposition information associated with the respective segmented anatomical region; and
a contrast of the respective segmented anatomical region as a function of image data of each voxel of the respective segmented anatomical region.

17. The CT system of claim 14, wherein further instructions are stored in the non-transitory memory that when executed, cause the processor to display a colorized overlay superimposed on the contrast-optimized image, the colorized overlay showing healthy tissues of the patient in a first color, and diseased tissues of the patient in a second color, the colorized overlay based on material decomposition data generated from the CT scan.

18. The CT system of claim 14, wherein further instructions are stored in the non-transitory memory that when executed, cause the processor to generate an automated report comparing the contrast-optimized image with a second contrast-optimized image, the second contrast-optimized image generated from previous scan data stored in a picture archiving and communications system (PACS) coupled to the CT system, the automated report showing a progression of a disease in an anatomical region of the patient, the progression of the disease determined by comparing a first size of a first area of diseased tissue in the contrast-optimized image with a second size of a second area of diseased tissue in the second contrast-optimized image.

19. A method for visualizing a progression of a disease of a patient, the method comprising:
performing a scan of the patient using a computed tomography (CT) system, and reconstructing a first image from projection data acquired during the scan;
adjusting display parameter settings of the CT system individually for each of a plurality of anatomical regions of the patient, to generate a first contrast-optimized image showing each anatomical region of the plurality of anatomical regions in an ideal contrast, the ideal contrast based on an assessed organ perfusion status of a contrast agent at the anatomical region;
retrieving a second image of the patient from a picture archiving and communications system (PACS) coupled to the CT system;
adjusting the display parameter settings of the CT system individually for each of the plurality of anatomical regions of the patient in the second image, to generate a second contrast-optimized image showing each anatomical region of the plurality of anatomical regions in the ideal contrast;
comparing a first size of a first area of diseased tissue in the first contrast-optimized image with a second size of a second area of diseased tissue in the second contrast-optimized image to determine the progression of the disease;
generating a visualization showing the first contrast-optimized image side-by-side with the second contrast-optimized image, the visualization including a measured difference between the first size and the second size;
sending an automated report including the visualization to a user of the CT system.

20. The method of claim 19, wherein the first contrast-optimized image and the second contrast-optimized image include one or more superimposed colorized overlays showing healthy tissues of the patient in a first color, and diseased tissues of the patient in a second color, the one or more superimposed colorized overlays based on material decomposition data generated from the scan.
